(19) United States
(12) Patent Application Publication
(10) Pub. No.: US 2023/0020319 A1
(43) Pub. Date: Jan. 19, 2023

(54) COMPOSITIONS AND METHODS FOR OPTOCHEMICAL CONTROL OF MTOR SIGNALING AND MTOR-DEPENDENT AUTOPHAGY

(71) Applicant: The University of Hong Kong, Hong Kong (CN)

(72) Inventors: Weiping Wang, Hong Kong (CN); Tianyi Wang, Hong Kong (CN); Kaiqi Long, Hong Kong (CN)

(21) Appl. No.: 17/850,720

(22) Filed: Jun. 27, 2022

Related U.S. Application Data
(60) Provisional application No. 63/215,814, filed on Jun. 28, 2021.

(51) Int. Cl. A61K 41/00; A61K 31/53; A61K 31/4745; A61K 31/5377; A61K 47/54; A61P 35/00

(52) U.S. Cl. CPC A61K 41/0042; A61K 31/53; A61K 31/4745; A61K 31/5377; A61K 47/545; A61P 35/00

(57) ABSTRACT

Compositions and methods for selective mTOR inhibition and/or increase of autophagy in a tissue of a subject have been developed for the treatment of cancer. Caged mTOR inhibitor prodrugs including photo-cleavable protecting groups are provided for selective chemotherapy through an optochemical treatment system. Pharmaceutical compositions of mTOR inhibitors that are deactivated (caged) with a photo-removable protecting group to controllably block the inhibitory activity of the inhibitor are provided. The photo-removable group is cleavable upon exposure to light irradiation, releasing the active inhibitor of mTOR signaling and autophagy at the site of irradiation. An exemplary caged mTOR inhibitor prodrug is a caged OSI-027 prodrug having a DEACM moiety bound thereto (cOSI-027). The cOSI-027 is activated in the region of a tumor by removal of the DEACM protecting group by exposure to light at 420 nm.

### CROSS-REFERENCE TO RELATED APPLICATION

This application claims the benefit of and priority to U.S. Provisional Application No. 63/215,814, filed on Jun. 28, 2021, which is hereby incorporated herein by reference in its entirety.

### FIELD OF THE INVENTION

The invention is generally directed to anti-cancer treatment, and in particular, photo-activatable inhibition of mTOR and enhancement of autophagy as a controllable, localized therapy for treating cancer.

### BACKGROUND OF THE INVENTION

Mammalian target of rapamycin (mTOR) is a serine/threonine protein kinase which plays an important part in regulating cell metabolism, proliferation, and survival (Faivre, et al., Nature Reviews Drug Discovery 2006, 5(8), 671-688; Guertin, et al., Cancer Cell 2007, 12(1), 9-22; Meric-Bernstam, et al., J Clin Oncol 2009, 27(13), 2278-2287; and Wullschleger, et al., Cell 2006, 124(3), 471-484.) The PI3K/Akt/mTOR pathway is reported to be the most frequently dysregulated pathway in various types of cancer (Engelman, et al., Nature Reviews Genetics 2006, 7(8), 606-619; Liu, et al., Nature Reviews Drug Discovery 2009, 8(8), 627-644; Samuels, et al. Science 2004, 304(5670), 554-554). Therefore, the mTOR signaling pathway offers a suitable therapeutic target for various cancer types.

The mTOR protein regulates the downstream signaling pathways through combining with other proteins to form two macromolecular complexes, mTOR complex 1 (mTORC1) and mTOR complex 2 (mTORC2). mTORC1 plays an important role in nutrient and energy sensing as well as regulating protein metabolism. mTORC2 functions as a regulator of the actin cytoskeleton through stimulating F-actin stress fibers, paxillin, RhoA, Rac1, Cdc42, and protein kinase Cα (PKCα) (Dos, et al., Current Biology 2004, 14 (14), 1296-1302). The mTORC2 protein also phosphorylates Akt to influence cell proliferation and survival.

Autophagy is a process closely related with mTOR signaling pathway. As a highly conserved cellular degradative and recycling process, it has been implicated to regulate cellular homeostasis and influence multiple perspectives of human health and pathophysiology (Parzych, et al., Antioxid Redox Signal 2014, 20 (3), 460-473). For example, amino acid starvation can decrease the activity of mTORC1 signaling and induce formation of autophagosomes, which fuse with lysosomes to form autolysosomes for protein degradation and amino acid supply. Although the formation of autolysosomes is commonly an adaptive process to stress, such as starvation to promote cell survival, autophagy can also inhibit cell proliferation and mediate cell death, which is called the type II programmed cell death or autophagic cell death (Tsujimoto, et al., Cell Death & Differentiation 2005, 12 (S2), 1528-1534; Shimizu, et al. Nature Cell Biology 2004, 6 (12), 1221-1228). In addition, overactivation of autophagy may lead to exhaustive degradation of cellular organelles, which can lead to cell death (Kroemer, G, et al., Nature Reviews Molecular Cell Biology 2008, 9 (12), 1004-1010). Therefore, the mTOR signaling and the autophagy processes can provide targets for cancer therapeutic strategies.

Multiple mTOR inhibitors have been considered for use in anticancer therapy, including OSI-027 (Mateo, et al., Br J Cancer 114, 889-896 (2016)). Unlike the mTORC1 allosteric inhibitor rapamycin, OSI-027 can inhibit both mTORC1 and mTORC2 pathways through binding to ATP-competitive binding site on the mTOR protein.

It is known that mTORC1 inhibitors, such as rapamycin, can induce hyperactivation of Akt through the negative feedback loop between S6K1 and IRS-1, which may lead to chemotherapeutic resistance (O'Reilly, et al., Cancer Res 2006, 66 (3), 1500-8). However, OSI-027 can be effective for rapamycin-insensitive cancer treatment, because of its ability to completely block the PI3K/Akt/mTOR pathway. The PI3K/Akt/mTOR pathway extensively affects protein and lipid synthesis, cytoskeleton organization and cell survival. In addition, autophagy was reported to play an important part in mediating OSI-027-induced cell death (Bhagwat, et al., Mol Cancer Ther 2011, 10 (8), 1394-406; O'Connor, et al., Cancer Research 2011, 71 (8 Supplement), 4463-44630). Therefore, OSI-027 exhibits a stronger suppression of cell proliferation and a higher anti-cancer potential than many other candidate drugs.

Unfortunately, OSI-027 therapy induces significant adverse side effects. As reported in the latest phase I clinical trial (NCT00698243), although 29.7% patients had stable disease in the 8-12-week follow-up assessment and six of these patients were free of progression for more than 24 weeks, OSI-027 induced dose-limiting toxicities (DLT) and adverse effects. According to the trial observations, the drug induced fatigue, gastrointestinal disorders, decreased cardiac function and cardiomyopathy, bone pain, and hyperglycemia. Importantly, OSI-027 therapy also frequently led to renal dysfunction, which may be due to the influence of mTOR inhibition on glomerular permeability and acute tubular necrosis (Mateo, et al., British Journal of Cancer 2016, 114 (8), 889-896). Thus, there is a need to improve the specificity of OSI-027 as a therapeutic drug, and to significantly reduce or avoid the adverse effects associated with mTOR inhibition as an anti-cancer therapy.

Therefore, it is an object of the invention to provide compositions and methods of use thereof for treatment of cancers by highly selective, controllable inhibition of mTOR and enhancement of autophagy.

It is another object to provide compositions and methods for enhancing the specificity of mTOR inhibitors.

It is a further object of the invention to provide compositions and methods for reducing adverse effects associated with mTOR inhibitors for anticancer therapy.

It is another object to provide compositions and methods for controllable and localized increase in autophagy activities.

### SUMMARY OF THE INVENTION

Compositions and methods for selective inhibition of mTOR and/or enhancement of autophagy in a tissue of a subject have been developed. mTOR inhibitors bound to photo-cleavable protecting groups are provided for selective chemotherapy through an optochemical treatment system. mTOR inhibitors that are deactivated (caged) with a photo-removable protecting group to controllably block the inhibitory activity of the inhibitor are provided. The photo-removable group is cleaved by light irradiation to release the active inhibitor of mTOR signaling and cancer cell proliferation at the site of irradiation.

Pharmaceutical compositions for treating cancer in a subject, including an effective amount of a caged mTOR inhibitor prodrug are provided. The caged prodrug includes an mTOR inhibitor and a photo-removable caging moiety that prevents the activity of the inhibitor. The caging moiety is reversibly bound to the inhibitor and is removable upon exposure to light irradiation in vivo. In an exemplary form, the mTOR inhibitor is OSI-027. An exemplary photo-removable caging moiety is 7-(diethylamino)-4-(hydroxymethyl)-coumarin (DEACM). Further exemplary photo-removable caging moieties are shown in Formulae III-VIII.

Methods of treating cancer in a subject are also provided. The methods include the steps of administering to the subject an effective amount of a caged mTOR inhibitor prodrug, including an mTOR inhibitor and a photo-removable caging moiety that prevents the activity of the inhibitor, and irradiating the cancer with light to remove the photo-removable caging moiety from the mTOR. Typically, irradiating the cancer with light includes exposing one or more cancer cells in the subject to light, wherein the light interacts with the prodrug to inhibit mTOR and/or enhancement of autophagy in one or more cancer cells in the subject. In some forms, the light has a wavelength of more than 300 nm, for example, about 420 nm. The light is generally administered to the subject immediately, or up to 1 hour, 2, 3, 4, 5, 6, 8, 10, 12, 18, 24, 36, 48 or 60 hours, or up to 1, 2, 3, 4, 5, 6, or 7 days, or any combination thereof after administration of the prodrug to the subject. Typically, the caged mTOR inhibitor prodrug is administered to the subject systemically, and the subsequent irradiation of the subject with light is restricted to the tissue or organ that is the site of the tumor(s) in the subject. In some forms, the methods include administering one or more additional active agents or procedures to the subject. For example, in some forms, the methods include administering surgery or radiation therapy to the subject prior to, or after treatment with the caged mTOR inhibitor prodrug.

The compositions and methods are particularly effective for treating a cancer characterized by dis-regulation of the PI3K/AKT/mTOR pathway. Exemplary cancers that can be treated include skin cancer, breast cancer, ovarian cancer, uterine cancer, prostate cancer, testicular tumor, brain cancer, gastric cancer, esophagus cancer, lung cancer, liver cancer, and colon cancer. In a particular form, the cancer to be treated is melanoma. A preferred amount of a caged mTOR inhibitor prodrug, including an mTOR inhibitor and a photo-removable caging moiety that prevents the activity of the inhibitor, is effective to reduce tumor size, reduce tumor viability, reduce tumor burden, prevent metastasis, reduce, or prevent one or more symptoms of the cancer, or combinations thereof in the subject after activation of the prodrug. In some forms, the amount of the mTOR inhibitor administered in the form of a caged mTOR inhibitor prodrug is an amount that is toxic to the subject if administered systemically in the absence of the caging moiety.

In some forms, the methods include one or more steps to identify or monitor the cancer prior to administering the caged mTOR inhibitor prodrug, or after administering the caged mTOR inhibitor prodrug, or both. In further forms, the methods repeat the steps of administering the caged mTOR inhibitor prodrug and irradiating the cancer with light one or more times, to reduce tumor size, reduce tumor burden, reduce tumor viability, prevent metastasis, or a combination thereof at the same or different sites in the subject.

In a particular form, methods for treating cancer include administering to a subject with cancer an effective amount of a photo-activatable mTOR inhibitor prodrug including OSI-027 bound to 7-(diethylamino)-4-(hydroxymethyl)-coumarin (DEACM) that prevents the anti-mTOR activity of OSI-027 and irradiating the cancer with light having a wavelength of about 420 nm. In another form, methods for treating cancer include administering to a subject with cancer an effective amount of a photo-activatable mTOR inhibitor prodrug including OSI-027 bound to boron-dipyrromethane (BODIPY)-based moiety that prevents the anti-mTOR activity of OSI-027 and irradiating the cancer with light having a wavelength of about 650 nm. The effective amount is effective to reduce tumor size, reduce tumor burden, reduce tumor viability, prevent metastasis, to reduce or prevent one or more symptoms of the cancer, or a combination thereof at the same or different sites in the subject. The methods are particularly useful in the diagnosis, prognosis, selection of patients, and the treatment of skin cancers. In some forms, the methods are effective in treating skin cancers, including basal and squamous cell skin cancers, Merkel cell cancer and melanomas.

### BRIEF DESCRIPTION OF THE DRAWINGS

FIG. 1 is a schematic illustration of the light-controlled mTOR inhibition system. The inactive caged OSI-027 prodrug (cOSI-027) is cleaved to the active form of OSI-027 upon exposure to 420 nm light irradiation. The active OSI-027 then blocks mTORC1 and mTORC2 activity, resulting in decreased local cell proliferation and viability.

FIGS. 2A-2C show synthesis and validation of the photo-activatable caged OSI-027 prodrug (cOSI-027). FIG. 2A is a representation of the synthesis of cOSI-027. FIGS. 2B and 2C show the ¹H-NMR spectrum of OSI-027and of cOSI-027, respectively.

FIGS. 3A-3C are graphs showing UV-Vis fluorescence spectra. FIG. 3A is a graph showing absorbance (0-4 a.u.) over wavelength (250-500 nm) for each of DEACM, OSI-027 and cOSI-027, respectively. FIG. 3B is a graph showing absorbance (0-4 a.u.) over wavelength (250-500 nm) for cOSI-027, when exposed to 420 nm light at 70 mW/cm² for 0 (as prepared), 1, 3, 5, 7, or 10 min, respectively. FIG. 3C is a graph showing fluorescence intensity (0-3000 a.u.) over wavelength (400-600 nm) for cOSI-027, when exposed to 420 nm light at 70 mW/cm² for 0 (as prepared), 1, 3, 5, 7, or 10 min, respectively (excitation wavelength at 380 nm).

FIGS. 4A and 4B show the photocleavage of cOSI-027 by HPLC and quantitative analysis. FIG. 4A is a line graph showing the photocleavage of cOSI-027 by HPLC analysis, with 0-1000 mA.U. at the wavelength (342 nm) for cOSI-027 and for OSI-027 when exposed to 420 nm light at 70 mW/cm² for 0, 1, 3, 5, 7, 10, or 15 min, respectively. FIG. 4B is a graph showing remaining cOSI-027 (0-400 μM) and released OSI-027 (0-300 μM) over light irradiation time (0, 1, 3, 5, 7, 10, or 15 min, respectively).

FIG. 5 is a photomicrograph showing bands corresponding to each of p-S6K, S6K, p-Akt, Akt, mTOR, and GAPDH, respectively, from cells treated with each of DMSO (control), OSI-027, DEACM, and cOSI-027, respectively, each with (+) or without (−) exposure to light (420 nm, 70 mW/cm², 3 min).

FIGS. 6A-6B show results of Western blotting and quantification of autophagy. FIG. 6A is a photomicrograph showing bands corresponding to each of LC3-I, LC3-II, and GADPH in cells treated with each of DMSO (control), OSI-027, DEACM, and cOSI-027, respectively, each with (+) or without (−) exposure to light. FIG. 6B is a graph showing normalized LC3-II/LC3-I ratio (0-15) for each of the bands in FIG. 6A including DMSO, OSI-027, DEACM, and cOSI-027, each with or without exposure to light, respectively. Light irradiation was done with a 420 nm LED (70 mW/cm², 3 min).

FIGS. 7A-7B are photomicrographs showing the presence of mCherry and/or GFP following exposure to different formulations and respective quantitative analysis. FIG. 7A is a panel of photomicrographs showing the presence of mCherry and/or GFP in cells treated with each of DMSO, OSI-027, DEACM, and cOSI-027, each with light irradiation or no light, respectively. FIG. 7B is a graph showing number of LC3 puncta per cell (0-300) for each of the groups in FIG. 7A. Light irradiation was done with a 420 nm LED (70 mW/cm², 3 min).

FIG. 8 is a bar graph showing cell viability (0%-150%) for cell groups treated with each of DMSO (control), OSI-027, DEACM, and cOSI-027, each at 50 μM, respectively, each with (+) or without (−) exposure to light. Light irradiation was done with a 420 nm LED (70 mW/cm², 3 min).

FIG. 9 is a graph showing the ¹H-NMR spectrum of OSI-027 caged by a red light-responsive BODIPY-based photo-removable protecting group.

FIGS. 10A-10C are graphs showing the photocleavage of OSI-027 caged by a red light-responsive BODIPY-based PPG by HPLC analysis. FIG. 10A shows the OSI-027 prodrug prior to light irradiation. FIG. 10B shows the OSI-027 prodrug after 656 nm light irradiation (100 mW/cm²) for 5 min, the peak of caged OSI-027 prodrug decreased significantly while free OSI-027 was released. FIG. 10C shows free OSI-027 analyzed by HPLC.

### DETAILED DESCRIPTION OF THE INVENTION

### I. Definitions

The terms “mTOR inhibitor,” or “inhibitor of mammalian target of rapamycin,” or “mTOR antagonist” refer to a pharmaceutical substance that, when administered to a cell, specifically prevents or reduces the activity of the mammalian target of rapamycin (mTOR) gene product. mTOR is a member of the phosphatidylinositol 3-kinase-related kinase family of protein kinases, which functions to interact with other proteins and serves as a core component of two distinct protein complexes, mTOR complex 1 (mTORC1) and mTOR complex 2 (mTORC1) to regulate different cellular processes. mTORC1/mTORC2 dual inhibitors are designed to compete with ATP in the catalytic site of mTOR. They inhibit kinase-dependent functions of mTORC1 and mTORC2 and block the feedback activation of PI3K/AKT signaling.

The terms “optochemical control” or “optochemical mechanism” refer to the control of a pharmacophore structure that has been deactivated or “caged” with a photo-cleavable protecting group. The photo-cleavable protecting group is removed as desired by exposure to light irradiation having a wavelength that causes photochemical release of the caging agent.

The terms “photo-activation” or “photo-removal” or “photo-cleavage” refer to the process of the removal of a photo-removable protecting group from a pharmacophore structure that has been deactivated or “caged” with a photo-cleavable protecting group.

The term “photoactivatable prodrug” refers to a drug that can exhibit an effect on a recipient that has been deactivated or “caged” with a photo-cleavable protecting group. The drug becomes activated when the photo-cleavable protecting group is removed by exposure to light irradiation having a wavelength that causes photochemical release of the caging agent. Typically, the prodrug will not exhibit any effect on the recipient in the absence of light irradiation.

The terms “inhibit” or “reduce” in the context of inhibition, mean to reduce or decrease in activity and quantity. This can be a complete inhibition or reduction in activity or quantity, or a partial inhibition or reduction. Inhibition or reduction can be compared to a control or to a standard level. Inhibition can be measured as a % value, e.g., from 1% up to 100%, such as 5%, 10, 25, 50, 75, 80, 85, 90, 95, 99, or 100%. For example, compositions including mTOR inhibitors may inhibit or reduce mTOR activity in cancer cells of the recipient by about 10%, 20%, 30%, 40%, 50%, 75%, 85%, 90%, 95%, or 99% of the mTOR activity in the same cancer cells prior to the treatment, or that did not receive, or were not treated with the compositions. In some forms, the inhibition and reduction are compared according to the level of mRNAs, proteins, cells, or tissues in the recipient.

The terms “treating” or “preventing” mean to ameliorate, reduce or otherwise stop a disease, disorder or condition from occurring or progressing in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating, or palliating the disease state, and remission or improved prognosis. For example, an individual is successfully “treated” if one or more symptoms associated with cancer are mitigated or eliminated, including, but are not limited to, reducing and/or inhibiting rate of tumor cell proliferation/growth, increasing the quality of life of those suffering from the disease, decreasing the dose of other medications required to treat the disease, delaying the progression of the disease, and/or prolonging survival of individuals.

The term “combination therapy” refers to treatment of a disease or symptom thereof, or a method for achieving a desired physiological change, including administering to an animal, such as a mammal, especially a human being, an effective amount of two or more chemical agents or components to treat the disease or symptom thereof, or to produce the physiological change, wherein the chemical agents or components are administered together, such as part of the same composition, or administered separately and independently at the same time or at different times (i.e., administration of each agent or component is separated by a finite period of time from each other).

The term “dosage regime” refers to drug administration regarding formulation, route of administration, drug dose, dosing interval and treatment duration.

The terms “individual,” “host,” “subject,” and “patient” are used interchangeably, and refer to a mammal, including, but not limited to, murines, simians, humans, mammalian farm animals, mammalian sport animals, and mammalian pets.

The term “effective amount” or “therapeutically effective amount” refers to the amount that is able to treat one or more symptoms of cancer, reverse the progression of one or more symptoms of cancer, halt the progression of one or more symptoms of cancer, or prevent the occurrence of one or more symptoms of cancer in a subject to whom the formulation is administered, for example, as compared to a matched subject not receiving the compound. The actual effective amounts of compound can vary according to the specific compound or combination thereof being utilized, the particular composition formulated, the mode of administration, and the age, weight, condition of the individual, and severity of the symptoms or condition being treated.

The term “pharmaceutically acceptable” or “biocompatible” refers to compositions, polymers, and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions, or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient. The term “pharmaceutically acceptable salt” is art-recognized, and includes relatively non-toxic, inorganic, and organic acid addition salts of compounds. Examples of pharmaceutically acceptable salts include those derived from mineral acids, such as hydrochloric acid and sulfuric acid, and those derived from organic acids, such as ethanesulfonic acid, benzenesulfonic acid, and p-toluenesulfonic acid. Examples of suitable inorganic bases for the formation of salts include the hydroxides, carbonates, and bicarbonates of ammonia, sodium, lithium, potassium, calcium, magnesium, aluminum, and zinc. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; and N-benzylphenethylamine

### II. Compositions

It has been demonstrated that optochemical control of mTOR inhibition and/or enhancement of autophagy can substantially enhance the selectivity and specificity of mTOR inhibitors for clinical treatment of cancer with reduced toxicity. Compositions for optochemical control of mTOR inhibition with reduced side effects are provided.

Compositions of photoactivatable mTOR inhibitors include deactivated (caged) mTOR inhibitor prodrugs with a photo-cleavable protecting group. The compositions inhibit mTOR and/or increase autophagy at a site in a subject when exposed to a light source having a wavelength that can cleave the protecting group and release the active drug. A preferred photoactivatable mTOR inhibitor is caged OS1-027, including a DEACM photo-removable moiety.

### A. mTOR Inhibitors

The photoactivatable therapies include one or more agents that inhibit or reduce the activity of the mammalian target of rapamycin (mTOR) gene product in vivo.

mTOR is a member of the phosphatidylinositol 3-kinase-related kinase (PI3K) family of protein kinases, which plays an important part in regulating cell metabolism, proliferation, and survival. mTOR is a downstream effector of the PI3K/AKT pathway, and functions to interact with other proteins and serves as a core component of two distinct protein complexes, mTOR complex 1 (mTORC1) and mTOR complex 2 (mTORC2) to regulate different cellular processes. mTORC1 includes mTOR and two positive regulatory subunits, raptor and mammalian LST8 (mLST8), and two negative regulators, proline-rich AKT substrate 40 (PRAS40) and DEPTOR. mTORC2 includes mTOR, mLST8, mSinl, protor, rictor, and DEPTOR.

Typically, the mTOR inhibitor inhibits or reduces one or more activities of mTOR, PI3K, mTORC1, mTORC2, or the PI3K/Akt/mTOR pathway. For example, in some forms, the mTOR inhibitor inhibits or reduces some or all of the kinase-dependent functions of mTORC1 and mTORC2; decreases or prevents AKT phosphorylation on mTORC2, or on mTORC1, or both; blocks the feedback activation of PI3K/AKT signaling; decreases protein translation, attenuates cell cycle progression, inhibits angiogenesis, or a combination thereof in a subject when administered to the subject. In preferred forms, the mTOR inhibitor is a mTOR/PI3K dual inhibitor. For example, in some forms, the mTOR inhibitor inhibits mTORC1, mTORC2, and all the catalytic isoforms of PI3K. In a particularly preferred form, the mTOR inhibitor blocks tumor cell proliferation by inducing G1 arrest in the tumor cell; induces apoptosis in tumor cells; induces autophagy in tumor cells, or combinations thereof.

In some forms, the mTOR inhibitor is cytotoxic and/or gives rise to severe adverse side effects when administered to a subject with cancer in an amount effective to reduce or prevent tumor growth.

An exemplary mTOR inhibitor is a small molecule inhibitor of mTOR. In some forms, the mTOR inhibitor is rapamycin (also known as sirolimus), or cyclosporine A. In other forms, the mTOR inhibitor is a rapamycin derivative or analog (rapalog), such as temsirolimus (CCI-779), everolimus (RAD001), or ridaforolimus/deforolimus (also known as AP23573), umirolimus, zotarolimus. In other forms, the mTOR inhibitor is a “second generation” mTOR inhibitor, such as an ATP-competitive mTOR kinase inhibitor.

In a preferred form, the mTOR inhibitor is OSI-027.

### 1. OSI-027

In some forms, the mTOR inhibitor is the small molecule Trans-4-[4-amino-5-(7-methoxy-1H-indol-2-yl)imidazo[5,1-f][1,2,4]triazin-7-yl]-cyclohexanecarboxylic acid (OSI-027). OSI-027 can inhibit both the mTORC1 and mTORC2 pathways through binding to ATP-competitive binding site in mTOR protein. OSI-027 has been shown to completely block PI3K/Akt/mTOR pathway, which extensively affect protein and lipid synthesis, cytoskeleton organization and cell survival. OSI-027 is also used for rapamycin-insensitive cancer treatment. The structure of OSI-027 (CAS Number 936890-98-1) is shown in Formula I, below. OSI-027 is available from multiple commercial sources (e.g., Sigma Aldrich product ID: COMH95E954DD-250MG).

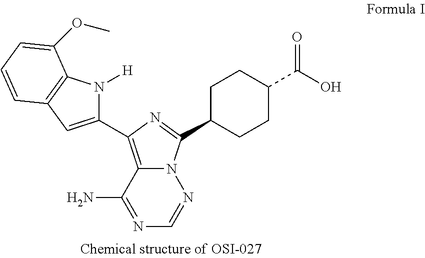 [SMILES: COc1cccc2cc(-c3nc([C@H]4CC[C@H](C(=O)O)CC4)n4ncnc(N)c34)[nH]c12]

Some other mTOR inhibitors that can be caged include, but are not limited to, Torin2 and PI103, which are also commercially available inhibitors for both mTORC1 and mTORC2.

### B. Photo-Removable (Caging) Moieties

The photoactivatable mTOR inhibitors include one or more moieties that deactivate (cage) the mTOR inhibitor with a photo-cleavable protecting group, which blocks its inhibitory effect on mTOR.

Photo-removable (sometimes called photo-releasable, photo-cleavable or photoactivatable) protecting groups (PPGs) provide spatial and temporal control over the release of various chemicals such as bioagents (enzymes, neurotransmitters, and cell-signaling molecules, etc). (Barltrop, et al., Tetrahedron Lett. 1962, 16, 697; Barton, et al., Tetrahedron Lett. 1962, 23, 1055; Barton, et al., J. Chem. Soc. 1965, Patchornik, et al., Am. Chem. Soc. 1970, 92, 6333; Sheehan, et al., J. Am. Chem. Soc. 1964, 86, 5277; and Engels, et al., J. Med. Chem. 1977, 20, 907). The term “caged” is used to designate a compound protected by a PPG.

In general, a PPG should have strong absorption at wavelengths well above 300 nm, where irradiation is less likely to be absorbed by (and possibly cause damage to) the biological entity. The photoreaction should occur with a high quantum yield or efficiency for release, Φrel. The quantum yield Φrel is equal to the amount of released substrate, nrel/mol, divided by the amount of photons at the irradiation wavelength λ, np/mol=np/einstein, that were absorbed by the caged compound: Φrel=nrel/np. An important measure for the efficacy of a PPG is the product of the quantum yield and the molar decadic absorption coefficient ε of the PPG, Φrelε(λirr), which is proportional to the amount of release at the given excitation wavelength (Klán, et al., Chemical Reviews 2013 113 (1), 119-191 DOI: 10.1021/cr300177k).

In preferred forms, the mTOR inhibitor is OSI-027, and the pharmacophore of OSI-027 is deactivated (caged) with a photo-cleavable protecting group, to block its mTOR inhibitory effect. Therefore, in some forms, the photo-removable group is cleaved after light irradiation to release the active OSI-027 to inhibit mTOR signaling and cancer cell proliferation. In a preferred form, the photoactivatable mTOR inhibitor is a caged OSI-027 prodrug (cOSI-027).

### 1. Photo-Removable 7-(diethylamino)-4-(hydroxymethyl)-coumarin (DEACM)

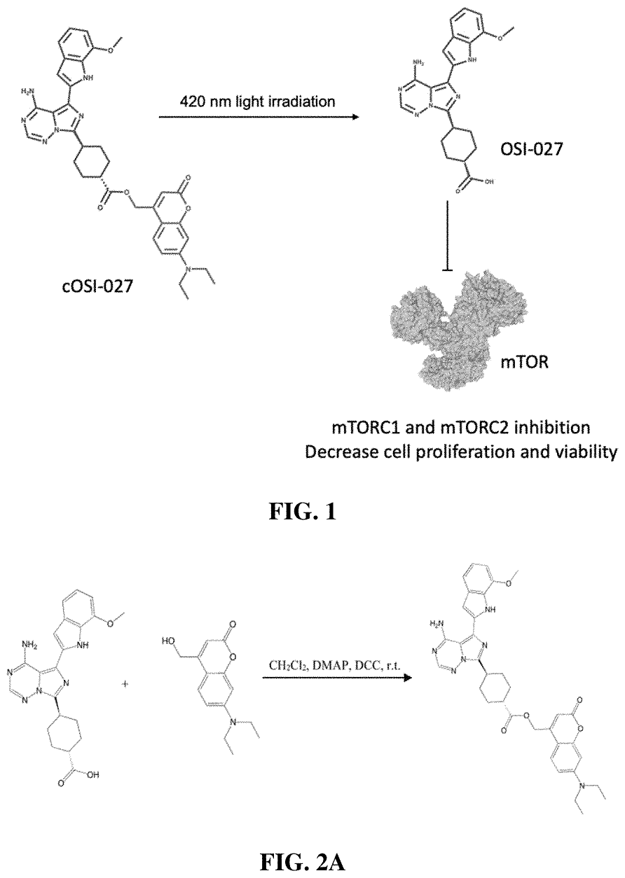

In particular forms, the caged OSI-027 prodrug is produced by masking the carboxyl group of OSI-027 with photo-cleavable 7-(diethylamino)-4-(hydroxymethyl)-coumarin (DEACM). DEACM has a fast photo-cleavable reaction and is applicable under visible light, for example, light having a wavelength of about 420 nm. Without light irradiation using a wavelength of about 420 nm, the caged OSI-027 prodrug will not exhibit inhibitory effect on mTOR. After 420 nm light irradiation, the photochemical released OSI-027 free drug prevents mTOR activity at the ATP-competitive catalytic site to inhibit phosphorylation of the mTORC1 and mTORC2 pathway, and to diminish protein anabolism and induce autophagy to impede the proliferation of the cells. A schematic representation of optochemical-controlled mTOR inhibition and autophagy induction is illustrated in FIG. 1. The chemical structure of 7-(diethylamino)-4-(hydroxymethyl)-coumarin (DEACM) is provided in Formula II, below.

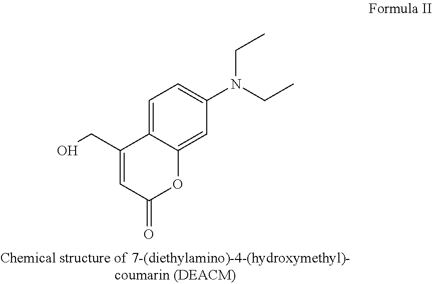 [SMILES: CCN(CC)c1ccc2c(CO)cc(=O)oc2c1]

7-(diethylamino)-4-(hydroxymethyl)-coumarin (Cas Number: 54711-38-5) is available from multiple commercial sources (e.g., Sigma Aldrich product ID: AMBH58064576-100MG).

Other photo-removable groups that can be used include, but are not limited to, 7-diethylamino-4-hydroxymethyl-thiocoumarin, 7-[bis(carboxymethyl)amino]-4-(hydroxymethyl) coumarin, 4-bromo-2-nitrobenzyl, 4,5-dimethoxy-2-nitrobenzyl, 1-(6-Nitrobenzodioxol-5-yl) ethanol, 1-(3-nitrodibenzofuran-2-yl)-ethanol, 1,3,5,7-tetramethyl -8-hydroxymethyl pyrromethene fluoroborate, 3-(4-methoxy)styryl-1,5,7-trimethyl-8-hydroxymethyl pyrromethene fluoroborate, 3-(4-dimethylamino)styryl-1,5,7-trimethyl-8-hydroxymethyl pyrromethene fluoroborate, 3,5-bisstyryl-1,7-dimethyl-8-hydroxymethyl pyrromethene fluoroborate, 3,5-Bis(4-methoxy)styryl-1,7-dimethyl-8-hydroxymethyl pyrromethene fluoroborate, 3,5-bis(4-dimethylamino)styryl-1,7-dimethyl-8-hydroxymethyl pyrromethene fluoroborate, 3,5-Bis(4-(1,4,7,10-tetraoxaundecyl)styryl-1,7-dimethyl-8-hydroxymethyl pyrromethene fluoroborate, 3,5-bis (4-methoxy)styryl-1,7-dimethyl-8-hydroxymethyl pyrromethene dimethylborate, 3,5-bis (4-dimethylamino)styryl-1,7-dimethyl-8-hydroxymethyl pyrromethene dimethylborate.

### 2. Photo-Cleavable boron-dipyrromethane (BODIPY)

Visible/NIR light absorbing boron-dipyrromethane (BODIPY) PPGs with easily tunable spectral properties and low phototoxicity can be used for non-invasive photo-release of protecting groups. Thus, in some forms, the PPGs are BODIPY-based PPGs. Chemical structures of BODIPY, or derivatives thereof are provided in Formulae III-VIII, below.

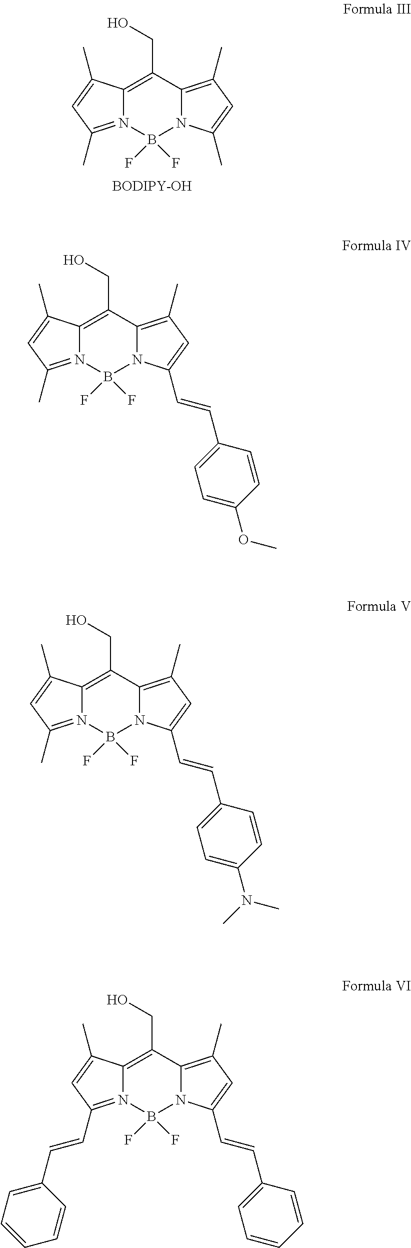

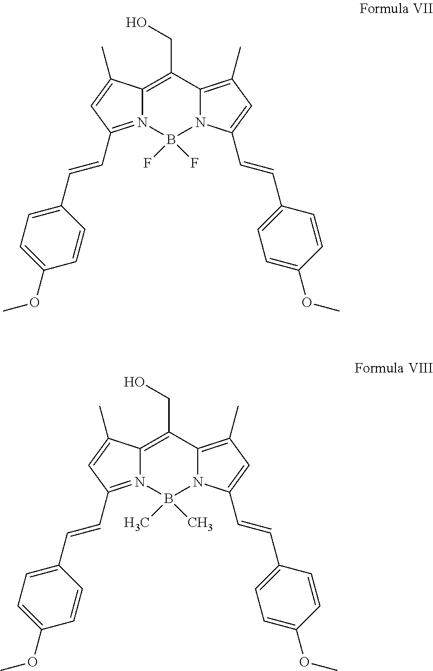

In particular forms, the caged OSI-027 prodrug is produced by conjugating to BODIPY, or derivatives thereof.

### C. Photoactivatable mTOR Inhibitor Prodrugs

Compositions of photoactivatable mTOR inhibitor prodrugs are provided. The inhibitor prodrugs include an mTOR inhibitor that is caged with a photo-removable moiety.

Typically, the caged mTOR inhibitor prodrug does not exhibit the same activity of the uncaged mTOR inhibitor in vivo prior to removal of the photo-removable moiety. For example, in some forms, the caged mTOR inhibitor prodrug does not inhibit or reduce one or more activities of mTOR, PI3K, mTORC1, mTORC2, or the PI3K/Akt/mTOR pathway; does not inhibit or reduce the kinase-dependent functions of mTORC1 and mTORC2; does not decrease or prevent AKT phosphorylation on mTORC2, or on mTORC1, or both; does not block the feedback activation of PI3K/AKT signaling; does not decrease protein translation, attenuate cell cycle progression, inhibit angiogenesis, or a combination thereof in a subject when administered to the subject. Typically, the caged mTOR inhibitor prodrug is not cytotoxic and/or does not give rise to severe adverse side effects when administered to a subject with cancer.

A preferred photoactivatable mTOR inhibitor is caged OS1-027, including a DEACM photo-removable moiety. The chemical structure of caged OSI-027 (cOSI-027) is provided in Formula IX, below.

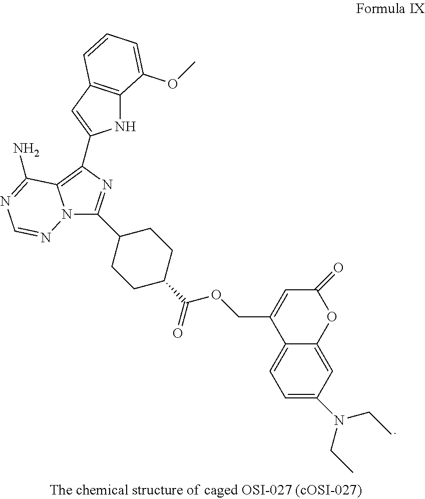

### D. Formulations

Formulations of, and pharmaceutical compositions including mTOR inhibitors having bound thereto photo-removable groups are provided. The formulations can include one or more mTOR inhibitors having bound thereto one or more photo-removable groups. In some forms, the pharmaceutical compositions include one or more additional active agents. Therefore, in some forms, the pharmaceutical composition includes two, three, or more active agents. The pharmaceutical compositions can be formulated as a pharmaceutical dosage unit, referred to as a unit dosage form.

Formulations typically include an effective amount of one or more mTOR inhibitors having bound thereto photo-removable groups. Effective amounts of the active agents are discussed in more detail below. It will be appreciated that in some forms the effective amount of an mTOR inhibitor having bound thereto a photo-removable group in a combination therapy is different from the amount that would be effective for the mTOR inhibitor having bound thereto a photo-removable group to achieve the same result when administered individually. For example, in some forms the effective amount of a mTOR inhibitor having bound thereto a photo-removable group is a lower dosage of the mTOR inhibitor having bound thereto a photo-removable group in a combination therapy than the dosage of the mTOR inhibitor having bound thereto a photo-removable group that is effective when administered alone.

### 1. Delivery Vehicles

The active agents or prodrugs thereof can be administered and taken up into the cells of a subject with or without the aid of a delivery vehicle. Appropriate delivery vehicles for the disclosed active agents are known in the art and can be selected to suit the particular agent. For example, in some forms, the active agent(s) is incorporated into or encapsulated by a nanoparticle, microparticle, micelle, synthetic lipoprotein particle, or carbon nanotube. For example, the compositions can be incorporated into a vehicle such as polymeric microparticles which provide controlled release of the active agent(s). In some forms, release of the drug(s) is controlled by diffusion of the active agent(s) out of the microparticles and/or degradation of the polymeric particles by hydrolysis and/or enzymatic degradation. Suitable polymers include ethylcellulose and other natural or synthetic cellulose derivatives. Polymers which are slowly soluble and form a gel in an aqueous environment, such as hydroxypropyl methylcellulose or polyethylene oxide may also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some forms, both agents are incorporated into the same particles and are formulated for release at different times and/or over different time periods. For example, in some forms, one of the agents is released entirely from the particles before release of the second agent begins. In other forms, release of the first agent begins followed by release of the second agent before all of the first agent is released. In still other forms, both agents are released at the same time over the same period of time or over different periods of time.

The active agent(s) can be incorporated into a delivery vehicle prepared from materials which are insoluble in aqueous solution or slowly soluble in aqueous solution but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including, but not limited to, fatty acid esters, fatty acid glycerides (mono-, di-, and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes.

Specific examples of waxes include beeswax, glycowax, castor wax, carnauba wax, paraffins and candelilla wax. As used herein, a wax-like material is defined as any material which is normally solid at room temperature and has a melting point of from about 30 to 300° C. The release point and/or period of release can be varied as discussed above.

### 2. Pharmaceutical Compositions

Pharmaceutical compositions including mTOR inhibitors having bound thereto photo-removable groups with or without a delivery vehicle are provided. Pharmaceutical compositions can be used for administration by parenteral (intramuscular, intraperitoneal, intravenous (IV) or subcutaneous injection), enteral, or transmucosal (nasal, vaginal, rectal, or sublingual) routes of administration or using bioerodible inserts and can be formulated in dosage forms appropriate for each route of administration.

In certain forms, the compositions are administered locally, for example, by injection directly into a site to be treated (e.g., into a tumor). In some forms, the compositions are injected or otherwise administered directly into the vasculature onto vascular tissue at the intended site of treatment. In some forms, the compositions are injected or otherwise administered directly into the vasculature onto vascular tissue adjacent to the intended site of treatment. Typically, local administration causes an increased localized concentration of the compositions which is greater than that which can be achieved by systemic administration. Targeting of the molecules or formulation can be used to achieve more selective delivery.

### a. Formulations for Parenteral Administration

mTOR inhibitors having bound thereto photo-removable groups and pharmaceutical compositions thereof can be administered in an aqueous solution, by parenteral injection. The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), antioxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria-retaining filter, by incorporating sterilizing agents into the compositions, by irradiating the compositions, or by heating the compositions.

### b. Enteral Formulations

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art. Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof. For example, in some forms, caged mTOR inhibitor prodrugs such as cOSI-027 set forth in Formula IX are administrated enterally.

### c. Extended Release, and Other Formulations

Alternatively, extended-release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form including single or multiple units. Examples of multiple units include, but are not limited to, multilayer tablets and capsules containing tablets, beads, or granules. An immediate release portion can be added to the extended-release system by means of either applying an immediate release layer on top of the extended release core using a coating or compression process or in a multiple unit system such as a capsule containing extended and immediate release beads.

Extended-release tablets containing hydrophilic polymers are prepared by techniques commonly known in the art, such as direct compression, wet granulation, or dry granulation processes. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin, and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from slippery solids such as talc, magnesium and calcium stearate, stearic acid, and hydrogenated vegetable oils.

Extended-release tablets containing wax materials are generally prepared using methods known in the art such as a direct blend method, a congealing method, and an aqueous dispersion method. In congealing method, the drug is mixed with a wax material and either spray-congealed or congealed and screened and processed.

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles, or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename EUDRAGIT® (Rohm Pharma; Westerstadt, Germany), including EUDRAGIT® L30D-55 and L100-55 (soluble at pH 5.5 and above), EUDRAGIT® L-100 (soluble at pH 6.0 and above), EUDRAGIT® S (soluble at pH 7.0 and above, as a result of a higher degree of esterification), and EUDRAGITS® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method, and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used Small quantities of an anti-foaming agent, such as a silicone (e.g., simethicone), may also be added to the coating composition.

Preferably, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another form, solvents that are low toxicity organic (i.e., non-aqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In one form, compositions may contain minor amounts of polymers, surfactants, or other excipients well known to those of the art. In this context, “minor amounts” means no excipients are present that might affect or mediate uptake of the compounds in the lungs and that the excipients that are present are present in amount that do not adversely affect uptake of compounds in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PARI Respiratory Equipment, Monterey, Calif.).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

### E. Adjunct and Additional Therapies and Procedures

Compositions of caged mTOR inhibitor prodrugs can be administered to a subject in combination with one or more adjunct therapies or procedures or can be an adjunct therapy to one or more primary therapies or producers. The additional therapy or procedure can be simultaneous or sequential with the mTOR inhibitors having bound thereto photo-removable groups. In some forms, the additional therapy is performed between drug cycles or during a drug holiday that is part of the dosage regime for mTOR inhibitors having bound thereto photo-removable groups. In preferred form, the additional therapy is a conventional treatment for cancer. For example, in some forms, the additional therapy or procedure is surgery, transplant surgery, a radiation therapy, or chemotherapy. For example, in a particular form mTOR inhibitors having bound thereto photo-removable groups are administered to a subject simultaneously or sequentially with a regime of a chemotherapeutic agent, e.g., Gemcitabine (Gemzar), Oxaliplatin (Eloxatin), Cisplatin, Doxorubicin (pegylated liposomal doxorubicin), Capecitabine (Xeloda), Mitoxantrone (Novantrone), docetaxel or cabazitaxel.

Representative chemotherapeutic agents include, but are not limited to, amsacrine, bleomycin, busulfan, camptothecin, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epipodophyllotoxins, epirubicin, etoposide, etoposide phosphate, fludarabine, fluorouracil, gemcitabine, hydroxycarb amide, idarubicin, ifosfamide, innotecan, leucovorin, liposomal doxorubicin, liposomal daunorubici , lomustine, mechlorethamine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, teniposide, tegafur-uracil, temozolomide, teniposide, thiotepa, tioguanine, topotecan, treosulfan, vinblastine, vincristine, vindesine, vinorelbine, vorinostat, taxol, trichostatin A and derivatives thereof, trastuzumab (HERCEPTIN®), cetuximab, and rituximab (RITUXAN® or MABTHERA®), bevacizumab (AVASTIN®), and combinations thereof. Representative pro-apoptotic agents include, but are not limited to, fludarabinetaurosporine, cycloheximide, actinomycin D, lactosylceramide, 15d-PGJ(2)5 and combinations thereof.

In some embodiments, the compositions and methods are used prior to or in conjunction with an immunotherapy such inhibition of checkpoint proteins such as components of the PD-1/PD-L1 axis or CD28-CTLA-4 axis using one or more immune checkpoint modulators (e.g., PD-1 antagonists, PD-1 ligand antagonists, and CTLA4 antagonists), adoptive T cell therapy, and/or a cancer vaccine. Exemplary immune checkpoint modulators used in immunotherapy include Pembrolizumab (anti-PD1 mAb), Durvalumab (anti-PDL1 mAb), PDR001 (anti-PD1 mAb), Atezolizumab (anti-PDL1 mAb), Nivolumab (anti-PD1 mAb), Tremelimumab (anti-CTLA4 mAb), Avelumab (anti-PDL1 mAb), and RG7876 (CD40 agonist mAb).

In some embodiments, the compositions and methods are used prior to or in conjunction with adoptive T cell therapy. Methods of adoptive T cell therapy are known in the art and used in clinical practice. Generally adoptive T cell therapy involves the isolation and ex vivo expansion of tumor specific T cells to achieve greater number of T cells than what could be obtained by vaccination alone. The tumor specific T cells are then infused into patients with cancer in an attempt to give their immune system the ability to overwhelm remaining tumor via T cells, which can attack and kill the cancer. Several forms of adoptive T cell therapy can be used for cancer treatment including, but not limited to, culturing tumor infiltrating lymphocytes or TIL; isolating and expanding one particular T cell or clone; and using T cells that have been engineered to recognize and attack tumors. In some embodiments, the T cells are taken directly from the patient's blood. Methods of priming and activating T cells in vitro for adaptive T cell cancer therapy are known in the art. See, for example, Wang, et al, Blood, 109(11):4865-4872 (2007) and Hervas-Stubbs, et al, J. Immunol., 189(7):3299-310 (2012).

Historically, adoptive T cell therapy strategies have largely focused on the infusion of tumor antigen specific cytotoxic T cells (CTL) which can directly kill tumor cells. However, CD4+ T helper (Th) cells such as Th1, Th2, Tfh, Treg, and Th17 can also be used. Th can activate antigen-specific effector cells and recruit cells of the innate immune system such as macrophages and dendritic cells to assist in antigen presentation (APC), and antigen primed Th cells can directly activate tumor antigen-specific CTL. As a result of activating APC, antigen specific Th₁ have been implicated as the initiators of epitope or determinant spreading which is a broadening of immunity to other antigens in the tumor. The ability to elicit epitope spreading broadens the immune response to many potential antigens in the tumor and can lead to more efficient tumor cell kill due to the ability to mount a heterogeneic response. In this way, adoptive T cell therapy can used to stimulate endogenous immunity.

In some embodiments, the T cells express a chimeric antigen receptor (CARs, CAR T cells, or CARTs). Artificial T cell receptors are engineered receptors, which graft a particular specificity onto an immune effector cell. Typically, these receptors are used to graft the specificity of a monoclonal antibody onto a T cell and can be engineered to target virtually any tumor associated antigen. First generation CARs typically had the intracellular domain from the CD3 ζ-chain, which is the primary transmitter of signals from endogenous TCRs. Second generation CARs add intracellular signaling domains from various costimulatory protein receptors (e.g., CD28, 41BB, ICOS) to the cytoplasmic tail of the CAR to provide additional signals to the T cell, and third generation CARs combine multiple signaling domains, such as CD3z-CD28-41BB or CD3z-CD28-OX40, to further enhance effectiveness.

In some embodiments, the compositions and methods are used prior to or in conjunction with a cancer vaccine, for example, a dendritic cell cancer vaccine. Vaccination typically includes administering a subject an antigen (e.g., a cancer antigen) together with an adjuvant to elicit therapeutic T cells in vivo. In some embodiments, the cancer vaccine is a dendritic cell cancer vaccine in which the antigen delivered by dendritic cells primed ex vivo to present the cancer antigen. Examples include PROVENGE® (sipuleucel-T), which is a dendritic cell-based vaccine for the treatment of prostate cancer (Ledford, et al., Nature, 519, 17-18 (5 Mar.2015). Such vaccines and other compositions and methods for immunotherapy are reviewed in Palucka, et al., Nature Reviews Cancer, 12, 265-277 (April 2012).

In some embodiments, the compositions and methods are used prior to or in conjunction with surgical removal of tumors, for example, in preventing primary tumor metastasis.

### III. Methods of Treatment

It has been established that optochemical control of mTOR inhibition can substantially enhance the selectivity and specificity of mTOR inhibitors for clinical treatment of cancer. Therapy involving targeted mTOR inhibition restricted to the area of a tumor using deactivated (caged) mTOR inhibitor with a photo-cleavable protecting group has been developed, to overcome adverse side effects associated with systemic mTOR inhibitors. The optochemically controlled therapy includes administration of an effective amount of a photoactivatable mTOR prodrug having a photo-removable protecting group to a subject in need thereof and exposing the subject to a light source having a wavelength that can cleave the protecting group and release the active drug.

Methods of treating a cancer or proliferative disease in a subject in need thereof are provided. The methods include administering to a subject one or more caged mTOR inhibitor prodrugs and subsequent exposure of the cancer to light having a wavelength effective to bring about photo-removal of the caging group from the mTOR inhibitor. The methods limit mTOR inhibition to the site of light exposure, thereby reducing the toxicity and adverse side effects associated with systemic mTOR inhibition. Therefore, the compositions and methods described herein enable enhanced specificity by targeted mTOR inhibition and enable increased localized dosing of mTOR inhibitors for killing of cancer cells.

Therapeutic treatment involves administering to a subject with cancer a therapeutically effective amount of an mTOR inhibitor in the form of a caged mTOR inhibitor prodrug, including an mTOR inhibitor having bound thereto a photo-removable caging moiety, and subsequent irradiation of the subject in the region of the cancer. In some forms the methods include administering to a subject in need thereof an effective amount of an mTOR inhibitor in the form of a caged mTOR inhibitor prodrug, including an mTOR inhibitor having bound thereto a photo-removable caging moiety, in combination with light irradiation to initiate localized mTOR inhibition and/or autophagy induction in the subject.

Methods for inducing and/or enhancing autophagy activities in one or more cancer cells are also provided. The methods include administering to a subject with cancer an effective amount of an mTOR inhibitor in the form of a caged mTOR inhibitor prodrug, including an mTOR inhibitor having bound thereto a photo-removable caging moiety, and irradiating the cancer to induce and/or enhance autophagy activities in the cancer cells. In some forms, the methods enhance autophagosome formation, autophagosome and autolysosome activity, and/or autophagic cell death compared to untreated control cancer cells.

Methods of treating one or more symptoms of cancer in a subject are provided. In certain forms, the methods include administering to a subject with cancer an effective amount of an mTOR inhibitor in the form of a caged mTOR inhibitor prodrug, including an mTOR inhibitor having bound thereto a photo-removable caging moiety, and irradiating the cancer to reduce or inhibit one or more symptoms of the cancer. In some forms, the methods administer an mTOR inhibitor in the form of a caged mTOR inhibitor prodrug, including an mTOR inhibitor having bound thereto a photo-removable caging moiety, in combination with one or more additional active agents to provide enhanced cancer treatment. For example, in some forms, the methods administer an mTOR inhibitor in the form of a caged mTOR inhibitor prodrug, including an mTOR inhibitor having bound thereto a photo-removable caging moiety, together with one or more additional chemotherapeutic agents, prior to exposing the cancer to a light source to induce mTOR inhibition and/or autophagy induction in the cancer. In preferred forms, the methods administer the mTOR inhibitor OSI-027 having bound thereto a photo-removable caging moiety prior to exposing the cancer to light having a wavelength of between about 350 nm and about 500 nm, inclusive. In preferred forms, the caged mTOR inhibitor is an OSI-027 prodrug, caged with photo-removable DEACM (cOSI-027), as set forth in Formula IX. In further preferred forms, the caged mTOR inhibitor is an OSI-027 prodrug, caged with photo-removable boron-dipyrromethane (BODIPY)-based moiety of any one of Formulae III-VIII. Typically, the methods decrease or inhibit the proliferation and/or viability of the cancer cells compared to untreated control cancer cells.

Methods for enhancing treatment of cancer by reducing adverse side effects and/or increasing the dose of mTOR inhibitors are also provided. For example, in some forms the methods treat a subject with cancer, wherein the subject is a subject to whom an mTOR inhibitor has previously been administered, and wherein the response achieved following the administration of a caged mTOR inhibitor prodrug, including an mTOR inhibitor having bound thereto a photo-removable caging moiety to the subject followed by irradiation of the cancer is greater than the response achieved by administering the mTOR inhibitor alone.

In some forms, the maximum dose of mTOR inhibitor that is administered to a subject is limited to sub-therapeutic quantities by side-effects and toxicity, having significant clinical impact on tumor treatment and prognosis. Therefore, in certain forms the methods are effective in providing therapeutic quantities of mTOR inhibitors and localized mTOR inhibition at the site of a cancer in the absence of systemic toxicity in a subject having cancer. Typically, the methods administer an effective amount of mTOR inhibitor in the form of a caged mTOR inhibitor prodrug, including an mTOR inhibitor having bound thereto a photo-removable caging moiety, to reduce or prevent one or more activities of mTOR, PI3K, mTORC1, mTORC2, or the PI3K/Akt/mTOR pathway. For example, in some forms the mTOR inhibitor is administered in the form of a caged mTOR inhibitor prodrug, including an mTOR inhibitor having bound thereto a photo-removable caging moiety, in an amount effective to inhibit or reduce some or all of the kinase-dependent functions of mTORC1 and mTORC2; decrease or prevent AKT phosphorylation and/or S6K phosphorylation on mTORC2, or on mTORC1, or both; block the feedback activation of PI3K/AKT signaling; decrease protein translation, attenuate cell cycle progression, inhibit angiogenesis, or a combination thereof in a tumor of a subject when exposed to light having a wavelength effective to remove the photo-removable caging moiety from the mTOR inhibitor prodrug.

The methods include steps of (1) administering to a subject an effective amount of a caged mTOR inhibitor prodrug, having bound thereto a photo-removable caging moiety, and (2) exposing one or more sites of the subject to light having a wavelength capable of causing removal of the photo-removable caging moiety from the mTOR inhibitor. In preferred forms, the methods administer the mTOR inhibitor OS1-027 in the form of a caged OS1-027 prodrug, including OS1-027 having bound thereto a photo-removable caging moiety. In a particularly preferred form, the mTOR inhibitor is caged OS1-027, including OS1-027 having bound thereto a photo-removable DEACM caging moiety (cOSI-027), as set forth in Formula IX. The light required to remove the photo-removable moiety DEACM should be between about 400 nm and 450 nm, preferably 420 nm light irradiation. In a further preferred form, the mTOR inhibitor is caged OS1-027, including OS1-027 having bound thereto a photo-removable boron-dipyrromethane (BODIPY)-based moiety of any one of Formulae III-VIII. The light required to remove the photo-removable dipyrromethane (BODIPY)-based moiety should be between about 600 nm and 750 nm, preferably 650 nm light irradiation.

### A. Selecting Patients for Combination Therapies

In some forms, the methods include one or more steps of identifying a subject in need of cancer therapy. Typically, the subject to be treated is one with one or more solid tumors. A solid tumor is an abnormal mass of tissue that usually does not contain cysts or liquid areas. Solid tumors may be benign (not cancer), or malignant (cancer). Examples of solid tumors are sarcomas, carcinomas, and lymphomas.

In some forms the methods characterize tumors and/or characterize the tumor microenvironment in a subject with cancer. For example, in some forms, the methods identify tumor cells and/or tumor infiltrating cells or tumor associated cells that are sensitive to therapy with mTOR inhibition and/or autophagy induction. Suitable methods of detection are known in the art. For example, in specific forms, the methods include a step of contacting the cell of the tumor with a molecule that immuno-specifically or physio-specifically binds proteins involved in or associated with the PI3K/Akt/mTOR pathway.

Methods for assessing the anti-cancer efficacy of mTOR inhibitors are also disclosed. In some forms the methods include steps of assessing tumor size and viability in a subject prior to and following administering mTOR inhibitors to the subject. For example, in some forms, tumor samples from cancer patients are characterized prior to and following treatment with mTOR inhibitors, in order to monitor changes in tumor size and viability as well as phenotypic and/or genetic changes within the tumor microenvironment.

In some forms, the subject is a subject that has cancer, and that is currently or has previously been administered mTOR inhibitors for cancer, and wherein the anti-cancer response achieved following the administration of the caged mTOR inhibitor prodrugs followed by light irradiation of the tumor is greater than the response achieved by administering the corresponding mTOR inhibitors alone. In some forms, the cancer may have developed a resistance to the maximum non-toxic dosage of mTOR inhibitors alone (i.e., when administered in the absence of the caged mTOR inhibitor prodrugs followed by light irradiation of the tumor). Therefore, in some forms, the subject population being treatment is defined as one in which the cancer being treated is resistant or insensitive to the maximum non-toxic dosage of mTOR inhibitors alone (i.e., when administered in the absence of the caged mTOR inhibitor prodrugs followed by light irradiation of the tumor).

### B. Methods of Administration and Dosage Regimes

The method and treatment regimens typically include treatment of a cancer, including administering to an animal, such as a mammal, especially a human being, an effective amount of a caged mTOR inhibitor prodrug, including an mTOR inhibitor having bound thereto a photo-removable caging moiety, and exposing one or more sites of the subject to light having a wavelength capable of causing removal of the photo-removable caging moiety from the mTOR inhibitor to treat the cancer.

The amount of a caged mTOR inhibitor prodrug, including an mTOR inhibitor having bound thereto a photo-removable caging moiety, present in a pharmaceutical dosage unit, or otherwise administered to a subject can be the amount effective to reduce the proliferation, viability, size, metastasis, or a combination thereof of the cancer cells when irradiated with light having a wavelength effective to remove the photo-removable caging moiety from the inhibitor. The amount of the active agents effective to decrease or inhibit the proliferation and/or viability of the cancer cells can be compared to untreated control cancer cells. In some forms, the amount of the active agents is effective to reduce, slow or halt tumor progression, or to reduce tumor burden in one or more tumors in the recipient, or a combination thereof. Tumor burden (tumor load—TB) is defined as the total amount of tumor (cells/mass) distributed in the patients' body, including bone marrow. In Response Evaluation Criteria in Solid Tumors (RECIST) analysis TB is considered the sum of the longest diameters of all measurable lesions. In some forms, the amount of the active agents is effective to alter a measurable biochemical or physiological marker. For example, in some forms, administration of a caged mTOR inhibitor prodrug, including an mTOR inhibitor having bound thereto a photo-removable caging moiety, decreases cancer cell viability by up to 10%, 20%, 30%, 40%, 50%, 60%, 70%, 80%, 90%, or up to 100% after a time (e.g., 12, 24, 36, 48, 60 or 72 hours) following light irradiation of the cancer cells. In an exemplary form, the administration of a caged mTOR inhibitor prodrug, including an mTOR inhibitor having bound thereto a photo-removable caging moiety, decreases cancer cell viability by up to 70%, 48 hours after light irradiation of the cancer cells, as set forth in the examples. An exemplary dosage of a caged mTOR inhibitor prodrug, including an mTOR inhibitor having bound thereto a photo-removable caging moiety, is about 0.1 mg/kg body weight, about 4 mg/kg body weight, about 7 mg/kg body weight, about 9 mg/kg body weight, about 11 mg/kg body weight, about 13 mg/kg body weight, and about 1,000 mg/kg body weight, inclusive.

A treatment regimen of the caged mTOR inhibitor prodrug, including an mTOR inhibitor having bound thereto a photo-removable caging moiety, can include one or multiple administrations of the caged mTOR inhibitor prodrug, and one or multiple irradiations with light. In preferred forms, the caged mTOR inhibitor prodrug is administered sequentially with administration of light irradiation/exposure of the tumor to light. For example, in some forms the caged mTOR inhibitor prodrug is administered at least 1, 2, 3, 4, 5, 6, 7, 8, 9, 10, 11, 12, 13, 14, 15, 16, 17, 18, 19, 20, 21, 22, 23, 24 or 30 minutes, hours or days prior to administering light irradiation to the subject in the area of the tumor.

In some forms, the methods also include steps to monitor the effects of the treatment. For example, in some forms the methods identify the suppression of a cancer's ability to grow. Suppression of a cancer can be measured using a biochemical assay, for example, measuring a decline in one or more biomarkers for the cancer in the blood, or by a morphometric analysis, for example by computerized tomography (CT), magnetic resonance imaging (MRI) or ultrasound. Therefore, in some forms the methods include the step of measuring the concentration of one or more biomarkers in the blood of the recipient. The measurements can be made before and after administration of the combination of a caged mTOR inhibitor prodrug and light irradiation to the subject.

In some forms the methods treat a cancer that has reacquired an ability to grow following prior treatment. For example, in some forms, the cancer has acquired resistance to one or more chemotherapies. A cancer that has reacquired an ability to grow can be an increase in tumor growth, the emergence, reemergence, or aggravation of symptoms, or new sites of metastasis.

In further forms, the methods are used for prophylactic use, i.e., prevention, delay in onset, diminution, eradication, or delay in exacerbation of signs or symptoms after onset, and prevention of relapse. For prophylactic use, a therapeutically effective amount of a the caged mTOR inhibitor prodrug, and subsequent targeted light irradiation is administered to a subject during early onset (e.g., upon initial signs and symptoms of cancer), or after an established development of cancer. Prophylactic administration can be used, for example, in the chemo-preventative treatment of subjects presenting precancerous lesions, and those diagnosed with early-stage malignancies.

### C. Cancers to be Treated

Methods of administering a caged mTOR inhibitor prodrug, and subsequent targeted light irradiation are effective treating multiple types of cancer.

Cancers that can be treated according to the described methods can be characterized by mutation of one or more genes, including cancers characterized as having one or more KRAS-mutations, HRAS-mutations, NRAS-mutations, EGFR mutations, ALK mutations, RB1 mutations, HIF mutations, KEAP mutations, NRF mutations, or other metabolic-related mutations, or combinations thereof.

In preferred forms, the compositions and methods are effective in treating one or more symptoms of cancers of the skin, lung, colon and rectum, bladder, etc. In particular forms, the methods are effective to reduce the size, viability, tumor burden, metastasis, or one or more symptoms of a skin cancer in the subject. The methods are effective against all types of skin cancer, including basal cell carcinoma, squamous cell carcinoma, Merkel cell cancer and melanoma.

The methods are also effective for treating other forms of cancer. Exemplary tumor cells that can be treated according to the described methods include, but are not limited to, tumor cells of cancers, including leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as, but not limited to, chronic myelocytic (granulocytic) leukemia, chronic lymphocytic leukemia, hairy cell leukemia; polycythemia vera; lymphomas such as, but not limited to, Hodgkin's disease, non-Hodgkin's disease; multiple myelomas such as, but not limited to, smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including, but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including, but not limited to, pheochromocytoma and adrenocortical carcinoma; thyroid cancer such as but not limited to papillary or follicular thyroid cancer, medullary thyroid cancer and anaplastic thyroid cancer; pancreatic cancer, including, but not limited to, insulinoma, gastrinoma, glucagonoma, vipoma, somatostatin-secreting tumor, and carcinoid or islet cell tumor; pituitary cancers including, but not limited to, Cushing's disease, prolactin-secreting tumor, acromegaly, and diabetes insipius; eye cancers including, but not limited to, ocular melanoma such as iris melanoma, choroidal melanoma, and ciliary body melanoma, and retinoblastoma; vaginal cancers, including, but not limited to, squamous cell carcinoma, adenocarcinoma, and melanoma; vulvar cancer, including, but not limited to, squamous cell carcinoma, melanoma, adenocarcinoma, basal cell carcinoma, sarcoma, and Paget's disease; cervical cancers including, but not limited to, squamous cell carcinoma, and adenocarcinoma; uterine cancers including, but not limited to, endometrial carcinoma and uterine sarcoma; ovarian cancers including, but not limited to, ovarian epithelial carcinoma, borderline tumor, germ cell tumor, and stromal tumor; esophageal cancers including, but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers including, but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including, but not limited to, hepatocellular carcinoma and hepatoblastoma, gallbladder cancers including, but not limited to, adenocarcinoma; cholangiocarcinomas including, but not limited to, papillary, nodular, and diffuse; lung cancers including, but not limited to, non-small cell lung cancer, squamous cell carcinoma (epidermoid carcinoma), adenocarcinoma, large-cell carcinoma and small-cell lung cancer; testicular cancers including, but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including, but not limited to, adenocarcinoma, leiomyosarcoma, and rhabdomyosarcoma; penal cancers; oral cancers including, but not limited to, squamous cell carcinoma; basal cancers; salivary gland cancers including, but not limited to, adenocarcinoma, mucoepidermoid carcinoma, and adenoidcystic carcinoma; pharynx cancers including, but not limited to, squamous cell cancer, and verrucous; skin cancers including, but not limited to, basal cell carcinoma, squamous cell carcinoma and melanoma, superficial spreading melanoma, nodular melanoma, lentigo malignant melanoma, acral lentiginous melanoma; kidney cancers including, but not limited to, renal cell cancer, adenocarcinoma, hypernephroma, fibrosarcoma, transitional cell cancer (renal pelvis and/or ureter); Wilms' tumor; bladder cancers including, but not limited to, transitional cell carcinoma, squamous cell cancer, adenocarcinoma, carcinosarcoma. Cancers that can be prevented, treated or otherwise diminished by the compositions include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, papillary adenocarcinomas, and gastric cancer (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

The light irradiation can be applied by shedding LED light or laser directly to the skin on top of tumor tissues. The light irradiation can also be applicable for deep tumor tissues, by shedding light from optical fibers or other possible devices into the tissues.

### IV. Kits

Medical kits are also disclosed. The medical kits can include, for example, a dosage supply of a caged mTOR inhibitor prodrug, supplied alone (e.g., lyophilized), or in a pharmaceutical composition. The active agent(s) can be in a unit dosage, or in a stock that should be diluted prior to administration. In some forms, the kit includes a supply of pharmaceutically acceptable carrier. The kit can also include devices for administration of the active agents or compositions, for example, syringes. The kits can include printed instructions for administering the compound in a method as described above.

The disclosed compositions and methods can be further understood through the following numbered paragraphs.

1. A pharmaceutical composition for treating cancer in a subject, comprising an effective amount of a caged mTOR inhibitor prodrug,

wherein the prodrug comprises

(a) an mTOR inhibitor; and

(b) a photo-removable caging moiety,

wherein the caging moiety is reversibly bound to the inhibitor, and

wherein the caging moiety prevents the activity of the inhibitor,

wherein the caging moiety is removable by exposure to light irradiation in vivo.

2. The pharmaceutical composition of paragraph 1, wherein the mTOR inhibitor is selected from the group consisting of OSI-027, Torin2, and PI103.

3. The pharmaceutical composition of paragraph 1 or 2, wherein the photo-removable caging moiety is selected from the group consisting of 7-(diethylamino)-4-(hydroxymethyl)-coumarin (DEACM), 7-diethylamino-4-hydroxymethyl-thiocoumarin, 7-[bis(carboxymethyl)amino]-4-(hydroxymethyl) coumarin, 4-bromo-2-nitrobenzyl, 4,5-dimethoxy-2-nitrobenzyl, 1-(6-Nitrobenzodioxol-5-yl) ethanol, 1-(3-nitrodibenzofuran-2-yl)-ethanol, 1,3,5,7-tetramethyl -8-hydroxymethyl pyrromethene fluoroborate, 3-(4-methoxy)styryl-1,5,7-trimethyl-8-hydroxymethyl pyrromethene fluoroborate, 3-(4-dimethylamino)styryl-1,5,7-trimethyl-8-hydroxymethyl pyrromethene fluoroborate, 3,5-bisstyryl-1,7-dimethyl-8-hydroxymethyl pyrromethene fluoroborate, 3,5-Bis(4-methoxy)styryl-1,7-dimethyl-8-hydroxymethyl pyrromethene fluoroborate, 3,5-bis(4-dimethylamino)styryl-1,7-dimethyl-8-hydroxymethyl pyrromethene fluoroborate, 3,5-Bis(4-(1,4,7,10-tetraoxaundecyl)styryl-1,7-dimethyl-8-hydroxymethyl pyrromethene fluoroborate, 3,5-bis(4-methoxy)styryl-1,7-dimethyl-8-hydroxymethyl pyrromethene dimethylborate, 3,5-bis(4-dimethylamino)styryl-1,7-dimethyl-8-hydroxymethyl pyrromethene dimethylborate.

4. The pharmaceutical composition of paragraph 1 or 2, wherein the photo-removable caging moiety has the structure of one of Formulae III-VIII:

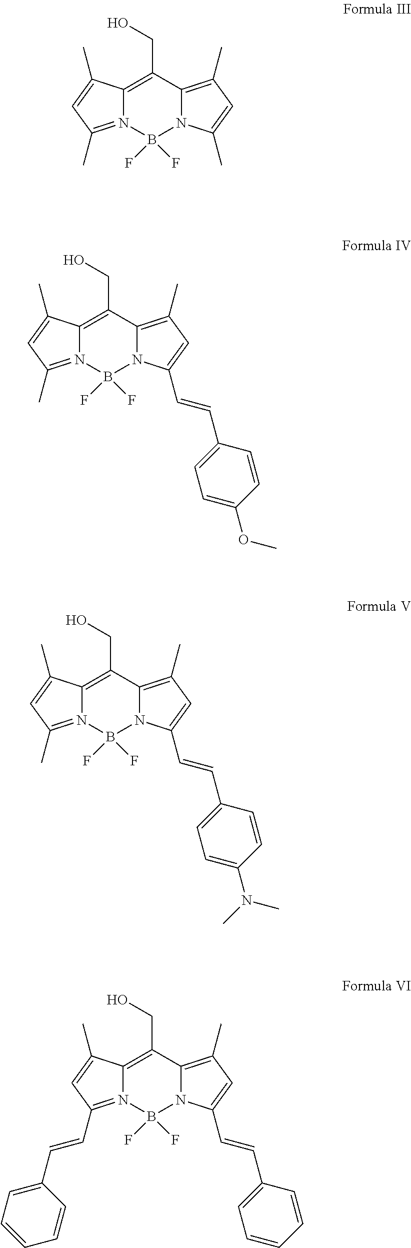

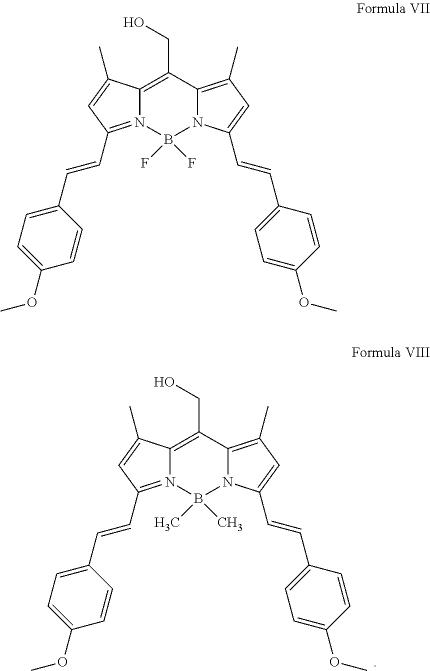

5. The pharmaceutical composition of paragraph 3 or 4, wherein the dosage of the prodrug is between about 0.1 mg/kg body weight and about 1,000 mg/kg body weight, inclusive.

6. A method of treating cancer in a subject, comprising

(a) administering to the subject an effective amount of the pharmaceutical composition of any one of paragraphs 1-5, and

(b) irradiating the cancer with light,

wherein administration of the light is effective to remove the photo-removable caging moiety from the mTOR inhibitor.

7. The method of paragraph 6, wherein irradiating the cancer with light comprises exposing one or more cancer cells in the subject to light, wherein the light interacts with the prodrug to inhibit mTOR and/or enhance autophagy in one or more cancer cells in the subject.

8. The method of paragraph 6 or 7, wherein the light has a wavelength of about 420 nm.

9. The method of any one of paragraphs 6 to 8, wherein the light is administered to the subject immediately, or up to 1 hour, up to 2, 3, 4, 5, 6, 8, 10, 12, 18, 24, 36, 48 or 60 hours, or up to 1 day, up to 2, 3, 4, 5, 6, or 7 days, or any combination thereof after administration of the prodrug to the subject.

10. The method of any one of paragraphs 6-9, further comprising administering one or more additional active agent(s) to the subject.

11. The method of any one of paragraphs 6-10, further comprising performing surgery or radiation therapy on the subject.

12. The method of any one of paragraphs 6-11, wherein the cancer to be treated is characterized by dis-regulation of the PI3K/AKT/mTOR pathway.

13. The method of any one of paragraphs 6-12, wherein the cancer is selected from the group consisting of skin cancer, breast cancer, ovarian cancer, uterine cancer, prostate cancer, testicular tumor, brain cancer, gastric cancer, esophagus cancer, lung cancer, liver cancer, and colon cancer.

14. The method of any one of paragraphs 6-13, wherein the mTOR inhibitor enhances autophagy activities in the cancer.

15. The method of any of paragraphs 6-14, wherein the effective amount is effective to reduce tumor size, reduce tumor burden, reduce tumor viability, prevent metastasis, or a combination thereof in the subject.

16. The method of any of paragraphs 6-15, wherein the amount of the mTOR inhibitor administered in the form of a caged mTOR inhibitor prodrug is an amount that is toxic to the subject if administered systemically in the absence of the caging moiety.

17. The method of any one of paragraphs 6-16, further comprising one or more steps to identify or monitor the cancer prior to administering the caged mTOR inhibitor prodrug, or after administering the caged mTOR inhibitor prodrug, or both.

18. The method of paragraph 17, further comprising repeating administration of the caged mTOR inhibitor prodrug and irradiating the cancer with light one or more times to reduce tumor size, reduce tumor burden, reduce tumor viability, prevent metastasis, or a combination thereof at the same or different sites in the subject.

19. A method for treating cancer, comprising

(a) administering to a subject with cancer an effective amount of an OSI-027 prodrug,

wherein the prodrug comprises OSI-027 bound to a photo-removable caging moiety that prevents the anti-mTOR activity of OSI-027, and

wherein the photo-removable caging moiety is 7-(diethylamino)-4-(hydroxymethyl)-coumarin (DEACM); and

(b) irradiating the cancer with light,

wherein the light has a wavelength of about 420 nm, and

wherein the effective amount is effective to reduce tumor size, reduce tumor burden, reduce tumor viability, prevent metastasis, to reduce or prevent one or more symptoms of the cancer, or a combination thereof in the subject.

20. A method for treating cancer, comprising

(a) administering to a subject with cancer an effective amount of an OSI-027 prodrug,

wherein the prodrug comprises OSI-027 bound to a photo-removable caging moiety that prevents the anti-mTOR activity of OSI-027, and

wherein the photo-removable caging moiety is a boron-dipyrromethane (BODIPY) -b as ed moiety; and

(b) irradiating the cancer with light,

wherein the light has a wavelength of about 650 nm, and

wherein the effective amount is effective to reduce tumor size, reduce tumor burden, reduce tumor viability, prevent metastasis, to reduce or prevent one or more symptoms of the cancer, or a combination thereof in the subject.

21. The method of any one of paragraphs 6-20, wherein the cancer is skin cancer selected from the group consisting of basal and squamous cell skin cancers, Merkel cell cancer and melanomas.

22. The method of any one of paragraphs 6-21, wherein the cancer is rapamycin-insensitive.

23. The method of any one of paragraphs 6-22, wherein the prodrug is administered systemically.

The disclosed compositions and methods can be further understood through the following examples.

### EXAMPLES

### Example 1: Synthesis of a Photoactivatable Caged OSI-027 Prodrug

A photoactivatable mTOR inhibitor, caged OSI-027 prodrug (cOSI-027), was designed by masking the carboxyl group of OSI-027 with photo-cleavable 7-(diethylamino)-4-(hydroxymethyl)-coumarin (DEACM), considering its fast photo-cleavable reaction and its applicability under visible light (420 nm). Without light irradiation, the prodrug does not exhibit inhibitory effects on the mTOR pathway.

After 420 nm light irradiation, the photochemical-released OSI-027 free drug can prevent the mTOR activity at the ATP-competitive catalytic site to inhibit phosphorylation of mTORC1 and mTORC2 pathway to diminish protein anabolism and induce autophagy to impede the proliferation of the cells. The optochemical-controlled mTOR inhibition and autophagy is depicted in FIG. 1.

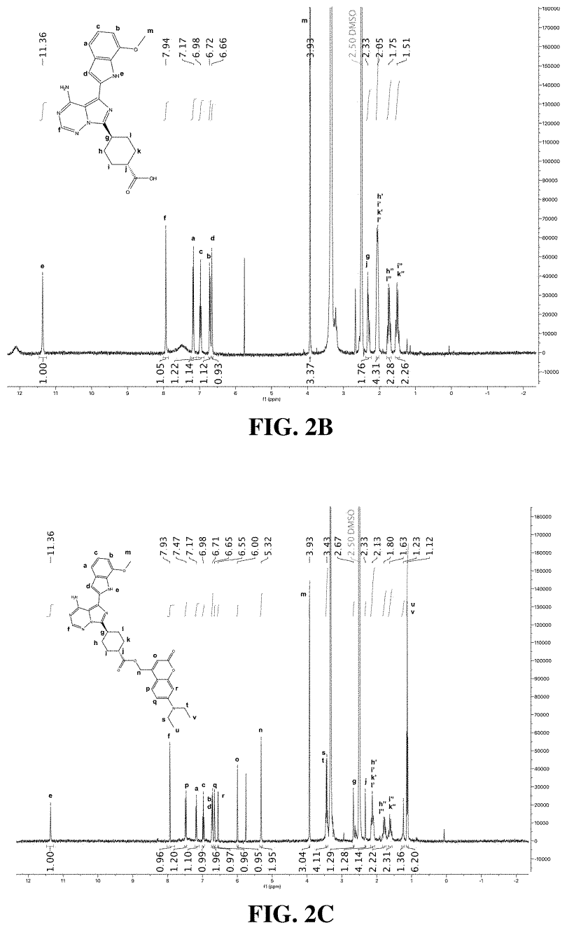

The synthesis route of the prodrug was shown in FIG. 2A. The OSI-027 and final product cOSI-027 were characterized by proton nuclear magnetic resonance (¹H-NMR) spectra (FIG. 2B and FIG. 2C).

### Example 2: Caged OSI-027 Prodrug is Cleaved to OSI-027 After Light Irradiation

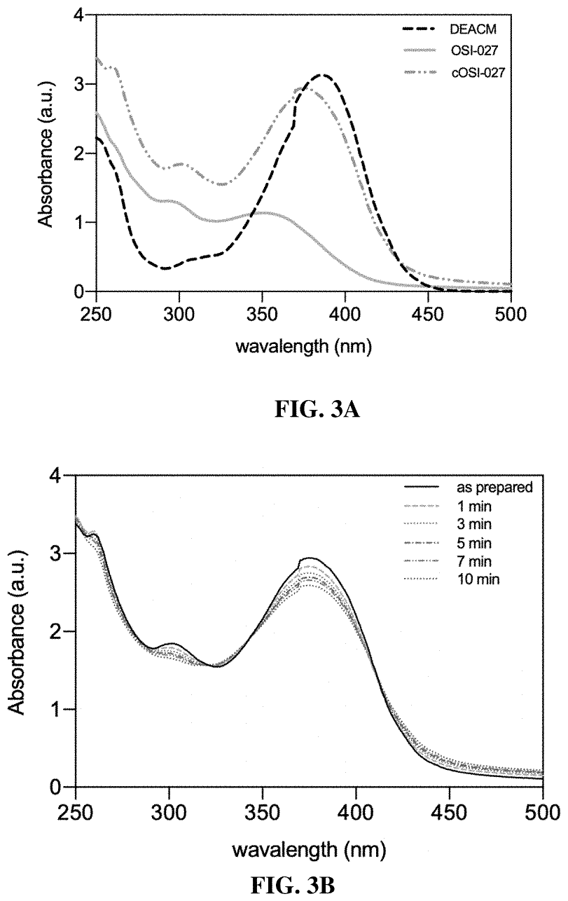

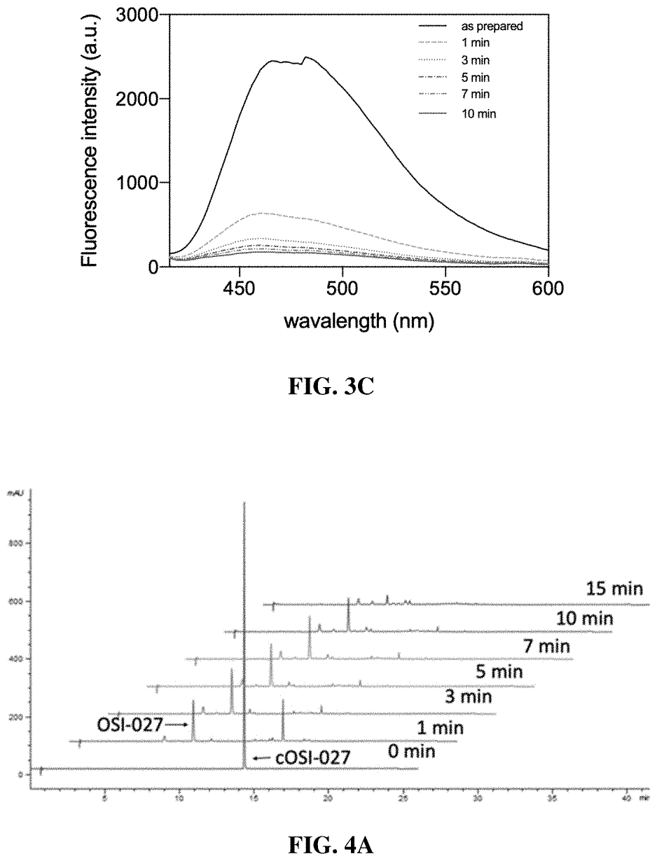

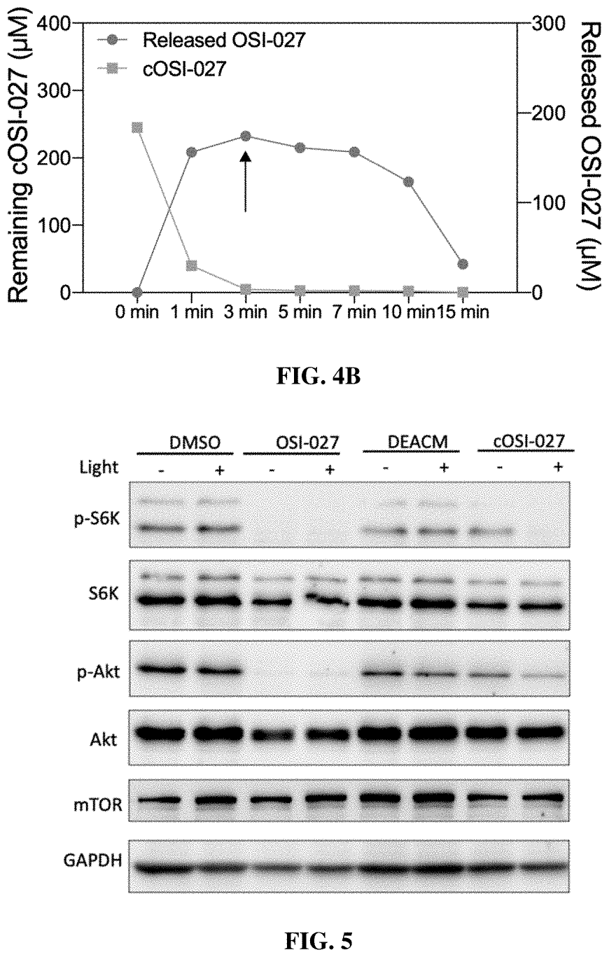

It was demonstrated that the caged OSI-027 prodrug is cleaved to OSI-027 after light irradiation. The optochemcial characteristics of the caged OSI-027 prodrug were analyzed by spectroscopy (FIGS. 3A-3C). The absorption peaks of the prodrug were located at 300 nm and 380 nm. The emission peak was located at 460 nm under 380 nm excitation. After 420 nm light irradiation, the prodrug showed both absorbance and emission spectrum change. To further verify the photocleavage reaction after light irradiation, high performance liquid chromatography (HPLC) was utilized to monitor the cleavage and release of OSI-027 after light irradiation. As shown in FIGS. 4A-4B, the generation of OSI-027 was characterized by the new peak at ˜8 min, after 1 min of 420 nm light irradiation. And the cleavage was almost completed after 3 min irradiation.

### Example 3: The mTOR Inhibitory Effect of cOSI-027 is Dependent Upon Light Irradiation

To further investigate the mTOR inhibitory effect of OSI-027 and cOSI-027 with or without light irradiation, western blot was performed to analyze the protein expression level of mTOR pathway (FIG. 5). S6K and Akt phosphorylation ratios were chosen as markers for mTORC1 and mTORC2 signaling pathway investigation, respectively. The western blot result showed that OSI-027 decreased the phosphorylation of S6K and Akt proteins compared with the control group, indicating its inhibitory effect on both mTORC1 and mTORC2 signaling pathway, respectively. cOSI-027 only exhibited significant inhibitory effect on mTORC1 and mTORC2 pathway after light irradiation.

### Example 4: The Autophagy-Enhancing Effect of cOSI-027 is Dependent Upon Light Irradiation

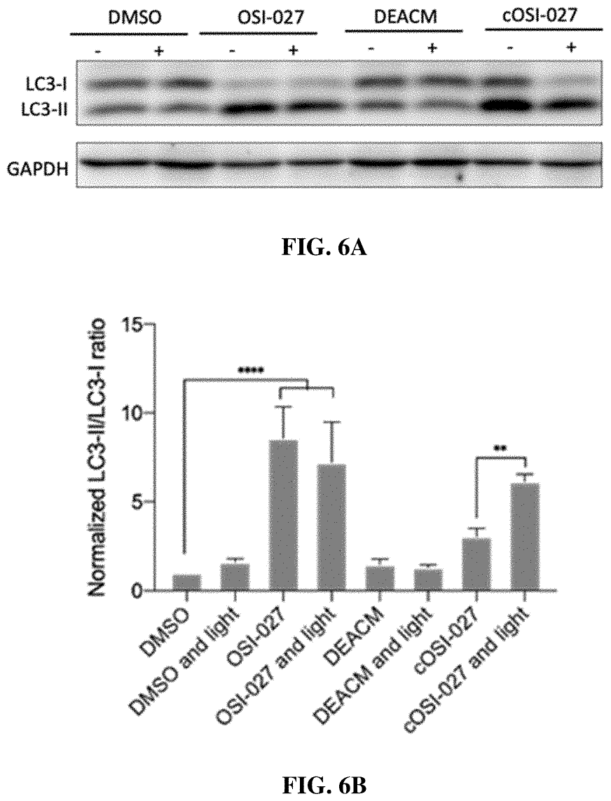

mTORC1 inhibition leads to higher autophagy activity, resulting in enhanced protein degradation ability in the lysosome. Therefore, whether the photochemical-controlled mTOR inhibition strategy can be used for manipulating autophagy activity was investigated. Autophagy level was characterized by the LC3-II/LC3-I ratio through western blot analysis (FIGS. 6A-6B). 24 h after the drug treatment, OSI-027 increased the LC3-II/LC-I ratio compared with the control group, indicating a higher autophagy activity. And cOSI-027 treatment led to a higher LC3-II/LC3-I ratio after light irradiation, compared with non-irradiation group.

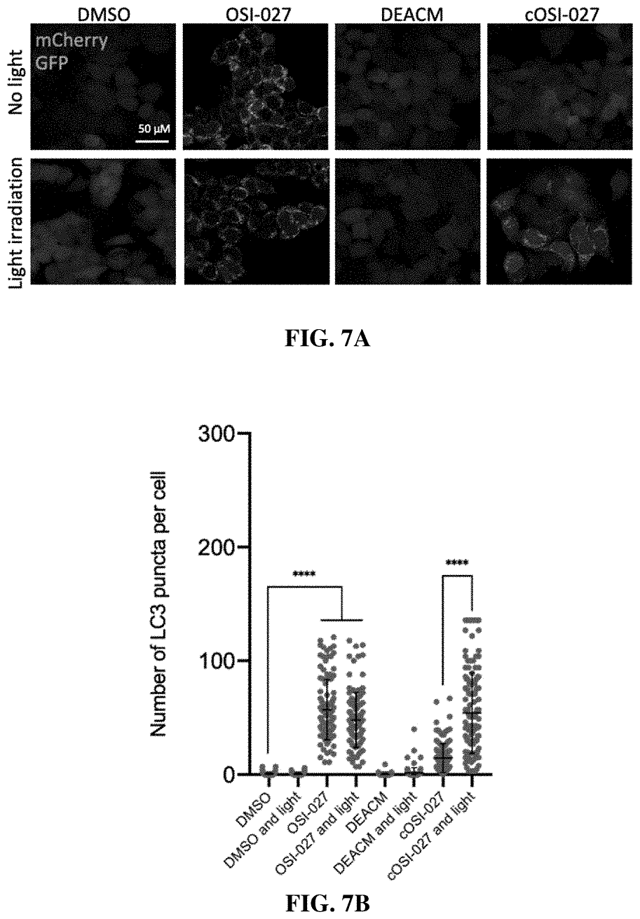

To further validate the photoactivated effect on autophagy, a mCherry-GFP-LC3 transfected HeLa cell line was established to accurately monitor the LC3 expression and subcellular localization. As shown in the confocal laser scanning microscopy (CLSM) images and the quantitative result (FIGS. 7A-7B), OSI-027 significantly increase the number and area of red and yellow puncta in the cells, compared with the control group, indicating higher autophagosome and autolysosome activity inside the cells. Likewise, cOSI-027 treatment increased the puncta number after light irradiation. These results indicated that this photo-activatable system can be used for triggering autophagy process in a mTOR-dependent manner

### Example 5: Photo-Activated OSI-027 has Anti-Cancer Activity

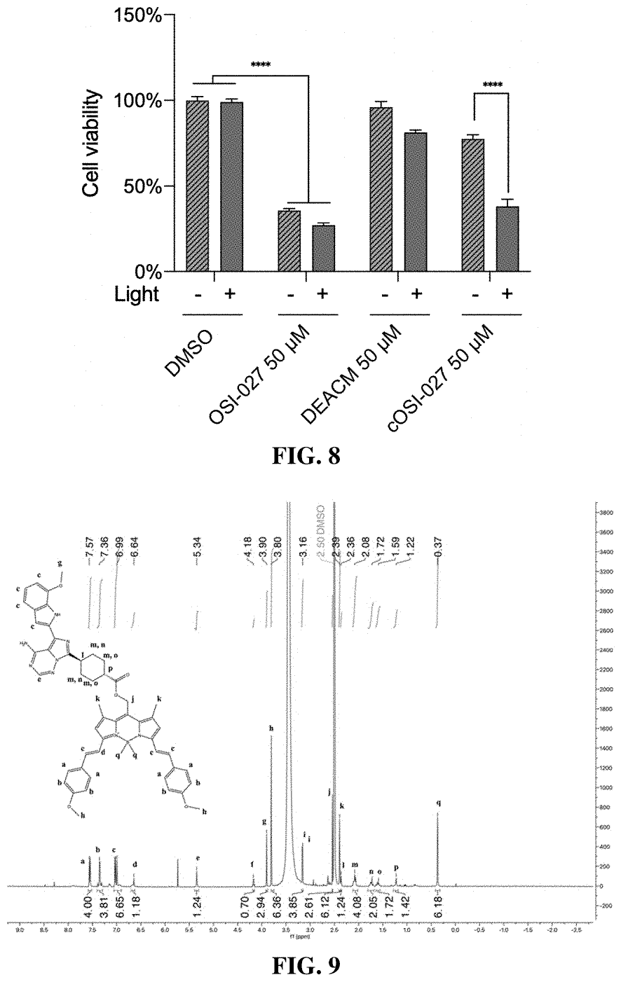

To investigate the anti-cancer effect of this photo-activatable prodrug system, MTT assay was further conducted to analyze the cell viability after the treatment with different formulations. As shown in FIG. 8, OSI-027 decreased the cell viability to ˜30% after 48 h incubation. And cOSI-027 also exhibited significantly lower cell viability after light irradiation, implying this light-controlled strategy can be used for anti-cancer treatment.

In summary, the photo-activatable prodrug, cOSI-027, allows the optochemically controlled mTOR inhibition with visible light irradiation, which can regulate both mTORC1 and mTORC2 pathways, induce mTOR-dependent autophagy, and lead to cancer cell death. This study demonstrates the first photoresponsive system of directly controlling both mTORC1 and mTORC2 signaling pathways and stimulating autophagy, which can both provide novel strategies for mTOR-targeting cancer therapy and act as a new research tool for further investigating mTOR signaling and its related biological processes.

### Example 6: OSI-027 Caged by a BODIPY-Based Photo-Removable Protecting Group

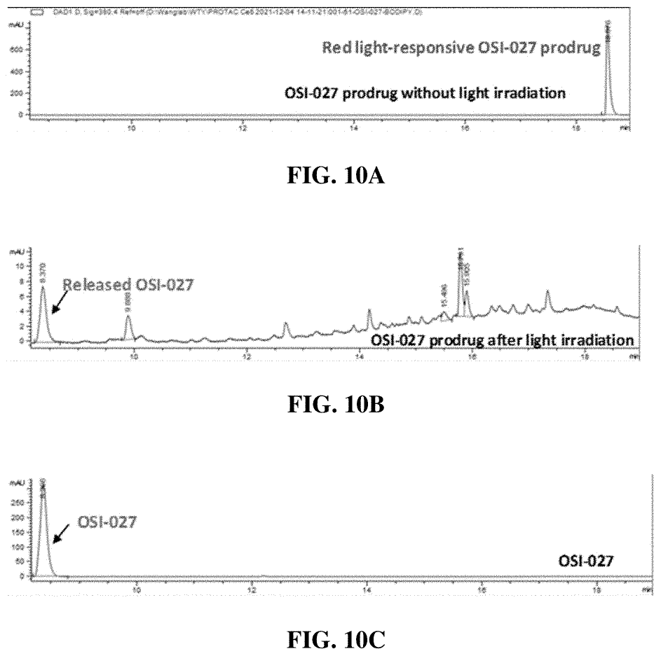

To further improve the clinical translation of the prodrug, we designed and synthesized another photocaged OSI-027 (FIG. 9). In this compound, OSI-027 was caged by a red light-responsive BODIPY-based photo-removable protecting group (PPG). The photolysis property was characterized by high-performance liquid chromatography (HPLC). The result showed that after 656 nm light irradiation (100 mW/cm²) for 5 min, the peak of caged OSI-027 prodrug decreased significantly while free OSI-027 was released (FIGS. 10A-10C). Therefore, in addition to our previous demonstration of blue light-responsive prodrug, red light-responsive OSI-027 prodrug can be designed to release free OSI-027 upon red-light irradiation, which may further facilitate the clinical application.

We claim:

1. A pharmaceutical composition for treating cancer in a subject, comprising an effective amount of a caged mTOR inhibitor prodrug, wherein the prodrug comprises
(a) an mTOR inhibitor; and
(b) a photo-removable caging moiety,
wherein the caging moiety is reversibly bound to the inhibitor, and
wherein the caging moiety prevents the activity of the inhibitor,
wherein the caging moiety is removable by exposure to light irradiation in vivo.

2. The pharmaceutical composition of claim 1, wherein the mTOR inhibitor is selected from the group consisting of OSI-027, Torin2, and PI103.

3. The pharmaceutical composition of claim 1, wherein the photo-removable caging moiety is selected from the group consisting of 7-(diethylamino)-4-(hydroxymethyl)-coumarin (DEACM), 7-diethylamino-4-hydroxymethyl-thiocoumarin, 7-lbis(carboxymethyl)aminol-4-(hydroxymethyl) coumarin, 4-bromo-2-nitrobenzyl, 4,5-dimethoxy-2-nitrobenzyl, 1-(6-Nitrobenzodioxol-5-yl) ethanol, 1-(3-nitrodibenzofuran-2-yl)-ethanol, 1,3,5,7-tetramethyl -8-hydroxymethyl pyrromethene fluoroborate, 3-(4-methoxy)styryl-1,5,7-trimethyl-8-hydroxymethyl pyrromethene fluoroborate, 3-(4-dimethylamino)styryl-1,5,7-trimethyl-8-hydroxymethyl pyrromethene fluoroborate, 3,5-bisstyryl-1,7-dimethyl-8-hydroxymethyl pyrromethene fluoroborate, 3,5-Bis(4-methoxy)styryl-1,7-dimethyl-8-hydroxymethyl pyrromethene fluoroborate, 3,5-bis(4-dimethylamino)styryl-1,7-dimethyl-8-hydroxymethyl pyrromethene fluoroborate, 3,5-Bis(4-(1,4,7,10-tetraoxaundecyl)styryl-1,7-dimethyl-8-hydroxymethyl pyrromethene fluoroborate, 3,5-bis(4-methoxy)styryl-1,7-dimethyl-8-hydroxymethyl pyrromethene dimethylborate, 3,5-bis(4-dimethylamino)styryl-1,7-dimethyl-8-hydroxymethyl pyrromethene dimethylborate.

4. The pharmaceutical composition of claim 1, wherein the photo-removable caging moiety has the structure of one of Formulae III-VIII:

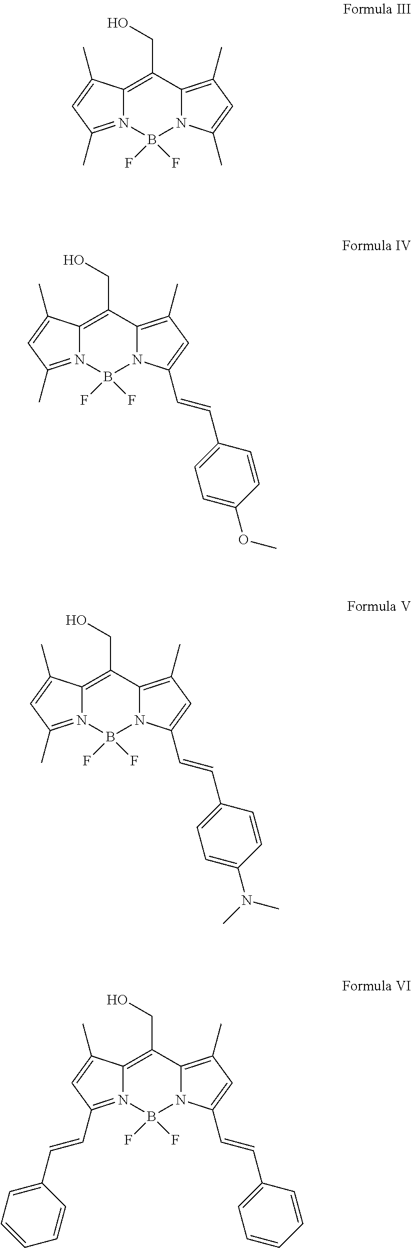

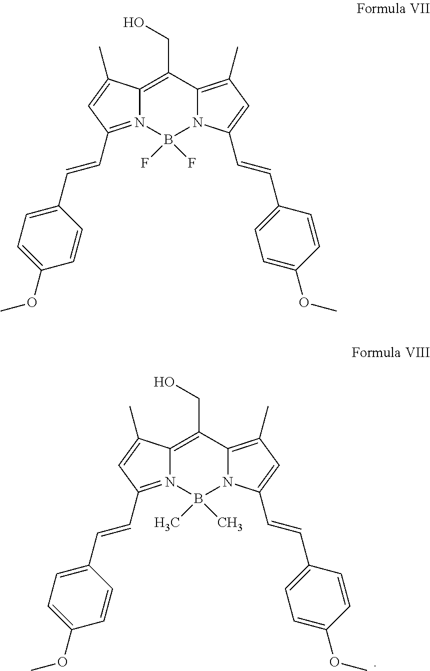

5. The pharmaceutical composition of claim 3, wherein the dosage of the prodrug is between about 0.1 mg/kg body weight and about 1,000 mg/kg body weight, inclusive.

6. A method of treating cancer in a subject, comprising
(a) administering to the subject an effective amount of the pharmaceutical composition of claim 1, and
(b) irradiating the cancer with light,
wherein administration of the light is effective to remove the photo-removable caging moiety from the mTOR inhibitor.

7. The method of claim 6, wherein irradiating the cancer with light comprises exposing one or more cancer cells in the subject to light, wherein the light interacts with the prodrug to inhibit mTOR and/or enhance autophagy in one or more cancer cells in the subject.

8. The method of claim 6, wherein the light has a wavelength of about 420 nm.

9. The method of claim 6, wherein the light is administered to the subject immediately, or up to 1 hour, up to 2, 3, 4, 5, 6, 8, 10, 12, 18, 24, 36, 48 or 60 hours, or up to 1 day, up to 2, 3, 4, 5, 6, or 7 days, or any combination thereof after administration of the prodrug to the subject.

10. The method of claim 6, further comprising administering one or more additional active agent(s) to the subject.

11. The method of claim 6, further comprising performing surgery or radiation therapy on the subject.

12. The method of claim 6, wherein the cancer to be treated is characterized by dis-regulation of the PI3K/AKT/mTOR pathway.

13. The method of claim 6, wherein the cancer is selected from the group consisting of skin cancer, breast cancer, ovarian cancer, uterine cancer, prostate cancer, testicular tumor, brain cancer, gastric cancer, esophagus cancer, lung cancer, liver cancer, and colon cancer.

14. The method of claim 6, wherein the mTOR inhibitor enhances autophagy activities in the cancer.

15. The method of claim 6, wherein the effective amount is effective to reduce tumor size, reduce tumor burden, reduce tumor viability, prevent metastasis, or a combination thereof in the subject.

16. The method of claim 6, wherein the amount of the mTOR inhibitor administered in the form of a caged mTOR inhibitor prodrug is an amount that is toxic to the subject if administered systemically in the absence of the caging moiety.

17. The method of claim 6, further comprising one or more steps to identify or monitor the cancer prior to administering the caged mTOR inhibitor prodrug, or after administering the caged mTOR inhibitor prodrug, or both.

18. The method of claim 17, further comprising repeating administration of the caged mTOR inhibitor prodrug and irradiating the cancer with light one or more times to reduce tumor size, reduce tumor burden, reduce tumor viability, prevent metastasis, or a combination thereof at the same or different sites in the subject.

19. The method of claim 6, wherein the cancer is skin cancer selected from the group consisting of basal and squamous cell skin cancers, Merkel cell cancer and melanomas.

20. The method of claim 6, wherein the cancer is rapamycin-insensitive.

21. The method of claim 6, wherein the prodrug is administered systemically.

22. A method for treating cancer, comprising
(a) administering to a subject with cancer an effective amount of an OSI-027 prodrug,
wherein the prodrug comprises OSI-027 bound to a photo-removable caging moiety that prevents the anti-mTOR activity of OSI-027, and
wherein the photo-removable caging moiety is 7-(diethylamino)-4-(hydroxymethyl)-coumarin (DEACM); and
(b) irradiating the cancer with light,
wherein the light has a wavelength of about 420 nm, and
wherein the effective amount is effective to reduce tumor size, reduce tumor burden, reduce tumor viability, prevent metastasis, to reduce or prevent one or more symptoms of the cancer, or a combination thereof in the subject.

23. A method for treating cancer, comprising
(a) administering to a subject with cancer an effective amount of an OSI-027 prodrug,
wherein the prodrug comprises OSI-027 bound to a photo-removable caging moiety that prevents the anti-mTOR activity of OSI-027, and
wherein the photo-removable caging moiety is a boron-dipyrromethane (BODIPY)-based moiety; and
(b) irradiating the cancer with light,
wherein the light has a wavelength of about 650 nm, and
wherein the effective amount is effective to reduce tumor size, reduce tumor burden, reduce tumor viability, prevent metastasis, to reduce or prevent one or more symptoms of the cancer, or a combination thereof in the subject.
